(19) United States
(12) Patent Application Publication
(10) Pub. No.: US 2003/0165806 A1
(43) Pub. Date: Sep. 4, 2003

(54) POTASSIUM CHANNELS, NUCLEOTIDE SEQUENCES ENCODING THEM, AND METHODS OF USING SAME

(76) Inventor: MARK H. PAUSCH, ROBBINSVILLE, NJ (US)

Correspondence Address: FINNEGAN, HENDERSON, FARABOW, GARRETT & DUNNER, LLP, 1300 I STREET, NW, WASHINGTON, DC, 20005, US

(21) Appl. No.: 08/816,011

(22) PCT Filed: Oct. 25, 1995
(86) PCT No.: PCT/US95/14364

(51) Int. Cl. C12Q 1/00; C07H 21/04; C12N 1/18; C07K 14/47; C12P 21/02; C12N 5/06

(52) U.S. Cl. 435/4; 530/350; 536/23.5; 435/69.1; 435/320.1; 435/325; 435/254.2

(57) ABSTRACT

This invention relates generally to a new family of potassium channels. More particularly, the present invention relates to the cloning and characterization of a family of distinct trans-membrane potassium ion channels, characterization of such channels, newly identified polynucleotide sequences, polypeptides encoded by such sequences, expression vectors capable of heterologous expression of such polynucleotide sequences, transformed host cells containing the expression vectors, and assay methods and kits therefor for determining the expression of heterologous nucleotide sequences encoding all or a portion of said potassium channels in host cells, chromosome mapping, diagnostic methodologies and kits therefore. Genes encoding potassium channels representative of this family were cloned from Drosophila melanogaster, Caenorhabditis elegans, human and mouse ESTs, and human brain, heart and kidney cDNA libraries. More particularly, the invention arises in part from the determination that the DNA sequences of these genes encode a structurally distinct potassium channel whose molecular architecture is characterized by four membrane spanning domains and two putative pore forming domains.

The application is a continuation-in-part of co-pending PCT/US95/14364 filed on Oct. 25, 1995 which is a continuation-in-part of U.S. Serial No. 332,312 filed on Oct. 31, 1994, now U.S. Pat. No. 5,559,026, issued Sep. 24, 1996.

### FIELD OF INVENTION

This invention relates generally to a new family of potassium channels. More particularly, the present invention relates to the cloning and characterization of a family of distinct trans-membrane potassium ion channels, characterization of such channels, newly identified polynucleotide sequences, polypeptides encoded by such sequences, expression vectors capable of heterologous expression of such polynucleotide sequences, transformed host cells containing the expression vectors and assay methods for determining the expression of heterologous nucleotide sequences encoding all or a portion of said potassium channels in host cells, chromosome mapping, diagnostic methodologies and kits therefor. Genes encoding potassium channels representative of this family were cloned from Drosophila melanogaster, Caenorhabditis elegans, human and mouse ESTs, and human brain, heart, and kidney cDNA libraries. More particularly, the invention arises in part from the determination that the DNA sequences of these genes encode a structurally distinct potassium channel whose molecular architecture is characterized by four membrane spanning domains and two putative pore forming domains.

### BACKGROUND OF THE INVENTION

Ion channels, which include sodium (Na⁺), potassium (K⁺), and calcium (Ca⁺⁺), are present in both eukaryotic and prokaryotic cells and control a variety of physiological and pharmacological processes. Potassium channels comprise a large and diverse group of integral membrane proteins that are involved in the movement of potassium into and out of the cell. Such channels regulate the level of excitability and repolarization properties of neurons and muscle fibers [B. Hille, Ionic Channels of Excitable Membranes, 2d Ed., Sinauer, Sunderland, Mass. (1992)] and are implicated in a broad spectrum of processes in both excitable and non-excitable cells. In almost all cells, K⁺ channels play a role in determining the resting electrical membrane potential by setting the membrane permeability to K⁺ ions. Potassium currents have been shown to be more diverse than sodium or calcium currents and play a role in determining the way a cell responds to external stimuli.

Several classes of K⁺ channels have been identified based on their pharmacological and electrophysiological properties; these include voltage-gated, ATP-sensitive, muscarinic-activated, S type, SK Ca⁺⁺-activated, Na⁺-activated, and inward and/or outward rectifier types of K⁺ channels. Prior to this work, and on the basis of membrane-spanning segments, potassium channels may be subdivided into topologically distinct classes. For example, one well-known class of voltage-gated, calcium activated, and/or cyclic nucleotide-gated-channels is composed of six membrane scanning domains (S1-S6) one of which contains repeated positive charges presumed to be involved in the voltage sensing of these channels and hence in their functional outward rectification and a single pore forming domain (H5 or P region). A second class may be described as an inward rectifying potassium channel that passes through the cellular membrane twice and also contains a single pore forming region [Y. Kubo, E. Reuveny, P. A. Slesinger, Y. N. Jan, L. Y. Jan, Nature 364, 802-806 (1993); Y. Kubo, T. J. Baldwin, Y. N. Jan, L. Y. Jan, Nature 362, 127-133 (1993); see also American Cyanamid copending U.S. patent application Ser. No. 08/431,928 filed on Jun. 28, 1995 for a description of “HIRK”].

The best characterized class of K⁺ channels are the voltage-gated outward rectifying channels (the Kᵥ family), the prototype being the protein which is coded for by the Shaker gene seen in Drosophila melanogaster, which is a voltage-gated channel. The proteins in this gene family contain a structural motif characterized by six membrane spanning segments (S1-S6), a putative voltage sensor (S4), and an S5-S6 linker (H5 or P region) involved in ion conductance. A functional channel is assembled in the membrane via the association of four Shaker subunits, necessitating the presence of four P domains.

Another well characterized class of potassium channel proteins, the inward rectifier potassium channels (Kᵢᵣ family) play a significant role in maintaining the resting potential of, and in controlling the excitability of a cell. These channels are characterized by two transmembrane domains and a pore-forming region and the lack of an S4 or voltage sensing region. Inward rectifying K⁺ channels are generally characterized by two transmembrane domains and one pore-forming domain. The pore-forming domain is common to both groups of K⁺ channels, the voltage-gated outward rectifier groups and the inward rectifying K⁺ channels and is an essential element of the aqueous K⁺-selective pore. A functional channel is assembled in the membrane via the association of four Kᵢᵣ subunits, necessitating the presence of four P domains.

A potassium channel from Saccharomyces cerevisiae designated Tok1, [Ketchum et al., Nature 376, 690-695 (1995)] or YORK [Lesage et al., J. Biol. Chem 271, 4183-4187 (1996)] has recently been identified and is characterized by the presence of two pore (2P) domains and an outward rectifying K⁺-selective current which is coupled to potassium equilibrium [Ketchum et al., Nature 376, 690-695 (1995)]. In contrast to the other channels described, the yeast channel comprises eight transmembrane domains, such domains resembling an assembly of an inward rectifying K⁺ channel of the Kᵢᵣ family (two transmembrane domains) with an outward rectifying channel of the Kᵥ family (six transmembrane domains).

A channel with four transmembrane domains and two pore-forming regions has recently been described by the present inventors [Goldstein, S. et al., Proc. Natl. Acad. Sci. USA 93 13256-13261 (1996)-“DmORF1” (also referred to as ORK1 or DORK)]. Other Investigators have described additional members of this potassium channel family [Fink, M. et al., EMBO J. 15, 6854-6862 (1996)-“TREK”; Lesage et al., EMBO Journal, 15, 1004-1011 (1996)-“TWIK-1”; Lesage F. et al., FEBS Lett. 402, 28-32 (1997)]. It has also been postulated that eight potassium channel families have been revealed by the C. elegans genome project, Wei A., et al., Neuropharmacology 35, No. 7, 805-829 (1996).

### SUMMARY OF THE INVENTION

A first aspect of the present invention is the discovery of a new family of potassium channel genes and proteins encoded thereby. Potassium channels belonging to this new family comprise four hydrophobic domains capable of forming transmembrane helices, wherein a first pore-forming domain is interposed between the first and second transmembrane helices and a second pore-forming domain is interposed between the third and fourth transmembrane helices, and the channels further contain various potassium selective peptide motifs. In preferred embodiments, the channels contain a GXG motif in the first pore-forming region and preferably in both pore-forming regions, wherein X is an amino acid selected from the group consisting of Y, F, V, I, M, and L, and particularly L or I. The channels preferably contain a further peptide motif in the P₁ and/or P₂ pore-forming regions, spanning several amino acids upstream of GXG, and particuarly for about six (6) amino acids upstream of the first G. Thus, the preferred pore-forming region motif is ZXXZ₁Z₂Z₃GXG where Z, Z₁ and Z₂ are preferably the amino acids residues T or S and Z₃ is preferably I or V, and X is as described above, again, with the amino acid residues L or I particularly preferred.

In further preferred embodiments, the channels display yet a second peptide motif, Z₄X₁X₂X₃GX₄PX₅, wherein Z₄ is the amino acid residue Y or F and preferably Y, and X₁, X₂, X₃, and X₄ are amino acid residues, wherein X₁ residues are A, S, or G, with A or S preferred; and X₂ through X₅ are the amino acid residues M, I, V, L, F, or Y, with L or I particularly preferred. In certain embodiments, this motif is “YALLGIP.” This second peptide motif is located downstream of Pl, generally about 12-25 amino acids downstream, and preferably about 16 amino acids downstream of P₁.

In certain preferred embodiments, the isolation and characterization of invertebrate (i.e. insect and nematode) potassium channel genes belonging to this new family is presented. In more preferred embodiments, the present invention further provides the isolation and characterization of polynucleotides from invertebrates and vertebrates, which encode amino acid sequence elements unique to this potassium gene family and specifically sourced from Drosophila melanogaster, Caenorhabditis elegans, avian libraries, murine and various other mammalian libraries, and libraries from all human tissues including human heart and brain.

A third aspect of the present invention is a method of controlling nematode and insect pests by inhibiting or activating potassium channels substantially homologous to those encoded by nucleotide sequences as presented herein. Another aspect of the present invention is to influence and alleviate human disease states modulating membrane potential with therapeutic agents that interact with the potassium channels biologically equivalent to those encoded by nucleotide sequences as encoded herein.

Various screening assay embodiments are also presented herein as well as chromosome identification and mapping techniques, diagnostic methodologies and kits therefore, and transgenic animals.

### BRIEF DESCRIPTION OF THE DRAWINGS

FIG. 1. Growth of CY162 cells bearing pDmORF1. CY162 cells transformed with plasmids isolated from survivors of a primary library screen for plasmids that support the growth of CY162 on medium contain low potassium concentration. Six individual transformants of each plasmid-bearing strain are cultured in patches on the indicated medium. CY162 cells bearing pDmORF1 are found in the upper left-hand corner of each plate while pKAT1 containing cells are found in the lower right hand corner.

FIGS. 2A and 2B. DNA sequence and deduced amino acid sequence of Dm ORF1 [SEQ ID NOS:1 and 2]. The nucleotide sequence of the 2.4 kb cDNA revealed a single long open reading frame proximal to the GAL1 promoter. Segments corresponding to putative transmembrane (M1-M4) and pore-forming H5 domains in the predicted polypeptide are underlined. The single amino-terminal asparagine linked glycosylation site is indicated by a G.

FIGS. 3A and 3B. DNA sequence and deduced amino acid sequence of the F22b7.7 segment of the Caenorhabditis elegans genome [SEQ ID NO:3]. Segments corresponding to putative transmembrane (M1-M4) and pore-forming H5 domains in the predicted polypeptide are underlined.

FIG. 4. Alignment of DmORF1 and F22b7.7 sequences. Protein-coding regions of DmORF1 [SEQ ID NO: 37] and F22b7.7 [SEQ ID NO: 381 (designated as CeORF-1 in this FIGURE) are compared using the protein sequence alignment algorithm in Genework DNA sequence analysis software. Identical amino acids are boxed.

FIG. 5A. Comparison of the pore-forming domains of DmORF1 and F22b7.7. Amino acid sequences from the six cloned Drosophila melanogaster potassium channels and three inward rectifier channels [SEQ ID NOS:7 through 21] are compared to DmORF1 and F22b7.7 within the pore-forming H5 regions. Amino acid identities are indicated by a vertical line and conserved substitutions indicated by a dot. Amino acid substitutions deemed acceptable are indicated.

FIG. 5B. Hydropathy plot analysis of the DmORF1 and F22b7.7 polypeptide sequence. The Kyte-Doolittle hydropathy algorithm in the Geneworks DNA analysis software is used to predict the topology of DmORF1 and F22b7.7. The position of predicted membrane spanning domains (M1-M4) and pore-forming domains are indicated.

FIG. 6. Predicted membrane spanning topology of DmORF1.

FIG. 7. Heterologous potassium channel-dependent growth of plasmid bearing CY162 (trkΔ) strains. CY162 bearing pYES2, pKAT1, pDmORF1, and pRATRAK are cultured at 30° C. for four days on arginine phosphate agar medium containing 0 mM, 0.2 mM, or 100 mM added KCl.

FIG. 8. Inhibition of growth of yeast cells containing heterologous potassium channels. CY162 cells (10⁵) bearing the indicated plasmids are plated in arginine phosphate agar medium containing 0.2 mM potassium chloride. Sterile filter disks were placed on the surface of the agar and saturated with 20 μl of a 1 M solution of potassium channel blocking compound. Clockwise from upper left-hand corner is BaCl₂, CsCl, TEA, and RbCl. KCl is applied to the center disk.

FIGS. 9A and 9B. DNA sequence and deduced amino acid sequence of CORK [SEQ ID NO: 36]. The nucleotide sequence of the 1.4 kb cDNA revealed a single long open reading frame proximal to the GAL1 promoter. Segments corresponding to pore-forming H5 domains in the predicted polypeptide are underlined. Asparagine-linked glycosylation sites are indicated by a G.

FIG. 10. Depicts a schematic representation of a preferred motif of the potassium channels of the invention.

### DETAILED DESCRIPTION OF THE INVENTION

Nucleotide bases are abbreviated herein as follows:

Ade; A-Adenine G-Guanine Ura; U-Uracil

C-Cytosine; T-Thymine; Ino; I or N (Inosine—bonds to any of the others)

Amino acid residues are abbreviated herein to either three letters or a single letter as follows:

Ala;A-Alanine Leu;L-Leucine

Arg;R-Arginine Lys;K-Lysine

Asn;N-Asparagine Met;M-Methionine

Asp;D-Aspartic acid Phe;F-Phenylalanine

Cys;C-Cysteine Pro;P-Proline

Gln;Q-Glutamine Ser;S-Serine

Glu;E-Glutamic acid Thr;T-Threonine

Gly;G-Glycine Trp;W-Tryptophan

His;H-Histidine Tyr;Y-Tyrosine

Ile;I-Isoleucine Val;V-Valine

The term “mammalian” as used herein refers to any mammalian species (e.g., human, mouse, rat, and monkey).

The term “heterologous” as used herein refers to nucleotide sequences, proteins, and other materials originating from organisms other than the host organism used in the expression of the potassium channels or portions thereof, or described herein (e.g., mammalian, avian, amphibian, insect, plant), or combinations thereof not naturally found in the host organism.

The terms “upstream” and “downstream” are used herein to refer to the direction of transcription and translation, with a sequence being transcribed or translated prior to another sequence being referred to as “upstream” of the latter.

The term “channel” and the nucleotide sequences encoding same, is intended to encompass all potassium channels, and mutants, derivatives, homologs, and other variations thereof.

The term “EST” as used herein refers to an expressed sequence tag.

Here we report the cloning and functional expression of a novel family of potassium channels exhibiting a unique topological configuration, and demonstrating particular physiological characteristics. Potassium channels belonging to this family may be derived from a wide variety of animal species, both vertebrate and invertebrate. This family is structurally and functionally novel, as manifested by the presence of two-pore forming domains (2P) in conjunction with a four membrane spanning domain configuration. Nucleotide sequences encoding various representative members of this new family of two-pore K⁺ channels were cloned by expression in yeast cells from Drosophila melanogaster (dORK or DmORF), and also by degenerate PCR from human brain, heart, and kidney cDNA (hORK1), and from human and mouse ESTs. Preliminary analyses of expression by a northern blotting procedure indicates that hORK1 is present primarily in human brain. Genes encoding structural homologues are present in the genome of Drosophila melanogaster (dORK), Caenorhabditis elegans (cORK), avian tissue and various mammalian tissue such as human (hORK1) and murine.

The potassium channel family of the present invention may be structurally characterized in that the potassium channels have four hydrophobic domains capable of forming transmembrane helices. These channels are further characterized in that they comprise two pore-forming domains, one of which is interposed between said first helix and said second helix, and the other of which is interposed between said third helix and said fourth helix. While the present inventors do not wish to be bound by theory, it is hypothesized that the 2P channels organize as dimers in the plasma membrane, consistent with a requirement for four (4P) domains to form a functional channel. The pore-forming domains further contain a potassium selective motif which serves to confer upon the channel the ability to pass potassium ions to the exclusion of other ions, such as sodium, calcium, and the like. In certain preferred embodiments, this motif contains the peptide Y/G, and particularly in either a dipeptide or tripeptide motif, and frequently with Y/F-G bonding. In more preferred embodiments, the motif comprises GXG, wherein X is an amino acid selected from the group consisting of V, L, Y, F, M, and I, and preferably L or I, such motif generally being found between the first two transmembrane domains. In certain other motif configurations, a second GXG motif, wherein X is an amino acid selected from the aforementioned group, is found between the third and fourth transmembrane domain as well. The channels preferably contain a further peptide motif in the P₁ and/or P₂ pore-forming regions, spanning several amino acids upstream of GXG, and particuarly for about six (6) amino acids upstream of the first G. Thus, the preferred pore-forming region motif is ZXXZ₁Z₂Z₃GXG where Z, Z₁ and Z₂ are preferably the amino acids residues T or S and Z₃ is preferably I or V, and X is as described above, again, with the amino acid residues L or I particularly preferred.

In yet further embodiments, the potassium channels of the invention comprise a second peptide motif, which in terms of the DNA encoding it, is located downstream of the first GXG motif, and within the second transmembrane domain (see FIG. 13 for a schematic depiction). This is the Z₄X₁X₂X₃GX₄PX₅ motif wherein Z₄ is the amino acid residue Y or F and preferably Y, and X is an amino acid residue wherein X₁ is A, S, or G with A or S preferred, and X₂ through X₅ are the amino acid residues M, I, V, L, F, or Y, with L or I particularly preferred. In other embodiments, the preferred Z₄X₁X₂X₃GX₄PX₅ motif is flanked by the first GXG motif (that is located between the first and second transmembrane domain) and is located in the second transmembrane, and a second pore-forming peptide motif is located downstream of the first pore-forming motif, between the third and fourth transmembrane domains. In preferred embodiments, the preferred Z₄X₁X₂X₃GX₄PX₅ motif is located downstream of the first pore-forming peptide motif by about 12-25 amino acids. In. other preferred embodiments the first pore-forming peptide motif is within about 16 amino acids. In general, the topological configuration of the potassium channels of the invention is such that one may presume that a regulatory domain of indeterminate length often may be interposed between the second transmembrane domain (TM2) and the third transmembrane domain (TM3). Thus, the size and characteristics of this domain may vary with cell type and needs, and is thereby a structure that is conducive to the conveyance of biological flexibility to the requirements and function of a particular cell. In certain embodiments, Z₄X₁X₂X₃GX₄PX₅ comprise the amino acids YALLGX₄P, and particularly “YALLGIP.”

In other embodiments, the potassium channels of the present invention further comprise a glycosylation site. This site may be an amino-terminal glycosylation site and may also be asparagine-linked.

The potassium channels of the present invention possess certain properties in common with known potassium channels including, voltage-gated channels, calcium activated channels, cyclic nucleotide gated channels, inward rectifier channels, and the like, and especially with regard to electrophysiological properties. However, a hallmark of the potassium channels of the invention are that they exhibit either outward current rectification or both inward and outward current rectification, in each case affected by potassium concentration.

Potassium channels play an essential role in determining the resting electrical membrane potential by setting the membrane permeability to K⁺ ions. The cloned 2P channels confer potassium selective currents when expressed in Xenopus oocytes. The dORK channels encode instantaneous open-pore channel activity. Thus, the potassium ions flow either into or out of the cell, depending on the magnitude and direction of the electrochemical driving force. In contrast, the human 2P channel designated herein as hORK1, is functionally distinguishable from dORK in that the hORK1 channel permits potassium flow primarily in an outward direction. Even when external potassium concentration is raised to the point where the electrochemical potential will drive potassium flux into oocytes containing dORK, little inward potassium current is observed in hORK1-containing oocytes.

It will be understood by those skilled in the art that the invention is not limited to the specific nucleotide and amino acid sequences depicted in the Sequence Listing, but also includes sequences that hybridize to such depicted sequences. Further, the invention also encompasses modifications to the depicted sequences, such as deletions, insertions, or substitutions in the sequence which produce changes in the resulting protein molecule that are not detrimental to the protein's activity. For example, alterations in the gene sequence which reflect the degeneracy of the genetic code, or which result in the production of a biologically equivalent amino acid at a given site, are contemplated; thus, a codon for the amino acid alanine, a hydrophobic amino acid, may be substituted by a codon encoding another less hydrophobic residue such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a biologically equivalent product. One skilled in the art will understand that assembly of 2P channel into functional dimers may require disulfide formation, and should take that into consideration when making modifications as taught herein [see e.g., Lesage et al., EMBO J. 15, 6400-6407 (1996)]. In some cases, it may in fact be desirable to make mutants of the sequence in order to study the effect of alteration on the biological activity of the protein. Each of the proposed modifications is well within the routine skill in the art, as is determination of the retention of biological activity of the encoded products.

The present invention further provides functional derivatives of the nucleotide sequences encoding the potassium channels of the invention. As used herein, the term “functional derivative” is used to define any DNA sequence which is derived from the original DNA sequence and which still possesses at least one of the biological activities present in the parent molecule. A functional derivative can be an insertion, deletion, or a substitution of one or more bases in the original DNA sequence.

Functional derivatives of the nucleotide sequences as presented herein, having an altered nucleic acid sequence can be prepared by mutagenesis of the DNA. This can be accomplished using one of the mutagenesis procedures known in the art. For example, preparation of functional derivatives may be achieved by site-directed mutagenesis. Site-directed mutagenesis allows the production of functional derivatives through the use of a specific oligonucleotide which contains the desired mutated DNA sequence. Site-directed mutagenesis typically employs a phage vector that exists in both a single-stranded and double-stranded form. Typical vectors useful in site-directed mutagenesis include vectors such as the M 13 phage, as disclosed by Messing et al., Third Cleveland Symposium on Macromolecules and Recombinant DNA, Editor A. Walton, Elsevier, Amsterdam (1981), the disclosure of which is incorporated herein by reference. These phage are commercially available and their use is generally well known to those skilled in the art. Alternatively, plasmid vectors containing a single-stranded phage origin of replication [Veira et al., Meth. Enzymol. 153:3 (1987)] may be employed to obtain single-stranded DNA.

While the site for introducing a sequence variation is predetermined, the mutation per se need not be predetermined. For example, to optimize the performance of a mutation at a given site, random mutagenesis may be conducted at a target region and the newly generated sequences can be screened for the optimal combination of desired activity.

Biologically equivalent refers to those modified nucleic acid and amino acid sequences in which the modified sequence at least substantially maintains the biological activity of the unmodified sequence; i.e., in the case of a nucleic acid sequence, the protein expressed therefrom at least substantially maintains the biological activity. Thus, the present invention also relates to the biologically equivalents of the potassium channel proteins whether specifically modified as described above or other isolated proteins. Biologically equivalent as used herein means protein having some homology with the hORK protein, wherein such protein maintains all or substantially all of the biological activity of the hORK protein, and contain the pore-forming peptide motif and preferably also the Z₄X₁X₂X₃GX₄PX₅ motif. The percentage of homology can vary from at least about 20% up to about 99.95%. Certainly percentage homologies of at least about 40%, at least about 70%, at least about 90% or at least about 95% can be employed based on the retention of biological activity. One skilled in this art will note that forty percent (40%) homology at amino acid level is usually consistent with retention of comparable 2° and 3° structure amongst homologs.

It is difficult to predict the exact effect of the substitution, deletion, insertion, or other modification in advance of making same, or to determine a suspected biological equivalent or functional derivative. However, one skilled in the art will recognize that the functionality of the modified construct or the suspected biological equivalent or functional derivative can be evaluated by routine screening assays. As one example, mRNA encoded by a functional derivative made by site-directed mutagenesis can be injected into an oocyte as described in the EXAMPLES and the oocyte tested for channel activity. Other target constructs may also be tested in this manner.

Any eukaryotic organism can be used as a source for a protein which is a member of the potassium channel family as described herein, or the genes encoding same, so long as the source organism naturally expresses such a protein or contains genes encoding same. As used herein, “source organism” refers to the original organism from which the amino acid or DNA sequence of the protein is derived, regardless of the organism the protein is expressed in and ultimately isolated from. For example, a member of the hORK family of channel proteins expressed in hamster cells, yeast cells, or the like, is of human origin as long as the amino acid sequence is that of a human protein which is a member of this family.

A variety of methodologies known in the art can be utilized to obtain a member of this family of channel proteins. In one method, the protein is purified from tissues or cells which naturally produce the protein. One skilled in the art can readily follow known methods for isolating proteins in order to obtain a member of the protein family, free of natural contaminants. These include, but are not limited to, immunochromatography, HPLC, size-exclusion chromatography, ion-exchange chromatography, and immunoaffinity chromatography.

The invention provides further methods of obtaining other members of this novel family of potassium channels, i.e., those sharing significant homology to one or more regions of the proteins described herein. Specifically, by using the sequences disclosed herein as probes or as primers, and techniques such as PCR cloning and colony/plaque hybridization, one skilled in the art can obtain other members of the family of potassium channel proteins as well as genomic sequences encoding such additional family members.

Region specific primers or probes derived from any of the sequences in the Sequence Listing can be used to prime DNA synthesis and PCR amplification, as well as to identify colonies containing cloned DNA encoding a member of this family using known methods.

When using primers derived from one of the nucleotide sequences for amplification, one skilled in the art will recognize that by employing high stringency conditions, annealing at 50°-60° C., sequences which are greater than 75% homologous to the primer will be amplified. By employing lower stringency conditions, annealing at 35°-37° C., sequences which are greater than 40-50% homologous to the primer will be amplified.

When using DNA probes derived from one of the nucleotide sequences for colony/plaque hybridization, one skilled in the art will recognize that by employing high stringency condition, hybridization at 50°-65° C., 5×SSPC, 0-50% formamide, wash at 50°-65° C., 0.5×SSPC, sequences having regions which are greater than 90% homologous to the probe can be obtained, and by employing lower stringency conditions, hybridization at 35°-37° C., 5×SSPC, 40-45% formamide, wash at 42° C., SSPC, sequences having regions which are greater than 35-45% homologous to the probe will be obtained.

Any tissue can be used as the source for the genomic DNA or RNA encoding members of the hORK family of potassium channels. However, with respect to RNA, the most preferred source is tissues which express elevated levels of the desired potassium channel family member. However, using the sequences as taught herein, it is now possible to identify such cells using the dORK, cORK or hORK sequence as a probe in northern blot or in situ hybridization procedures, thus eliminating the necessity to obtain RNA/DNA from a tissue which expresses elevated levels of such protein.

Genes encoding the potassium channels of the present invention may be expressed in a recombinant host. Heterologous DNA sequences are typically expressed in a host by means of an expression vector. An expression vector is a replicable DNA construct in which a DNA sequence encoding the heterologous DNA sequence is operably linked to suitable control sequences capable of affecting the expression of a protein or protein subunit coded for by the heterologous DNA sequence in the intended host. Generally, control sequences include a transcriptional promoter, an optional operator sequence to control transcription, a sequence encoding suitable mRNA ribosomal binding sites, and (optionally) sequences which control the termination of transcription and translation. Vectors useful for practicing the present invention include plasmids, viruses (including bacteriophage), and integratable DNA fragments (i.e., fragments integratable into the host genome by genetic recombination). The vector may replicate and function independently of the host genome, as in the case of a plasmid, or may integrate into the genome itself, as in the case of an integratable DNA fragment. Suitable vectors will contain replicon and control sequences which are derived from species compatible with the intended expression host. For example, a promoter operable in a host cell is one which binds the RNA polymerase of that cell, and a ribosomal binding site operable in a host cell is one which binds the endogenous ribosomes of that cell.

DNA regions are “operably associated” when they are functionally related to each other. For example, a promoter is operably linked to a coding sequence if it controls the transcription of the sequence; a ribosome binding site is operably linked to a coding sequence if it is positioned so as to permit translation. Generally, operably linked means contiguous and, in the case of leader sequences, contiguous and in reading phase.

Transformed host cells of the present invention are cells which have been transformed or transfected with the vectors constructed using recombinant DNA techniques and express the protein or protein subunit coded for by the heterologous DNA sequences. The novel nucleic acid sequences of the invention and fragments thereof can be used to express protein in a variety of host cells, both prokaryotic and eukaryotic. Examples of suitable eukaryotic cells include mammalian cells, plant cells, yeast cells, and insect cells. Suitable prokaryotic hosts include Escherichia coli and Bacillus subtilis. Illustrative of conventional mammalian host cells are chinese hamster ovary (CHO) cells, COS cells, human embryonic kidney cells, NIH3T3 fibroblasts and mouse Ltk cells. Illustrative of insect cells are SP9 cells.

Suitable expression vectors are selected based upon the choice of host cell. Numerous vectors suitable for use in transforming host cells are well known. For example, plasmids and bacteriophages, such as λ phase, are the most commonly used vectors for bacterial hosts, and for E. coli in particular. In both mammalian and insect cells, plasmid and virus vectors are frequently used to obtain expression of exogenous DNA. In particular, mammalian cells are commonly transformed with conventional viral vectors, or transfected with plasmids, such as the pcDNAI vector series from Invitrogen Corporation (San Diego, Calif.) and the pMAM vector series from Clontech, and insect cells in culture may be transformed with baculovirus expression vectors. Yeast vector systems include yeast centromere plasmids, yeast episomal plasmids and yeast integrating plasmids. The invention encompasses any and all host cells transformed or transfected by the claimed nucleic acid sequences or fragments thereof, as well as expression vectors used to achieve this.

In preferred embodiments, the transformed host cells are yeast. A variety of yeast cultures, and suitable expression vectors for transforming yeast cells, are known. See e.g., U.S. Pat. No. 4,745,057; U.S. Pat. No. 4,797,359; U.S. Pat. No. 4,615,974; U.S. Pat. No. 4,880,734; U.S. Pat. No. 4,711,844; and U.S. Pat. No. 4,865,989. Saccharomyces cerevisiae is the most commonly used among the yeasts, although a number of other yeast species are commonly available. See, e.g., U.S. Pat. No. 4,806,472 (Kluveromyces lactis and expression vectors therefore); 4,855,231 (Pichia pastoris and expression vectors therefore). A heterologous potassium channel may permit a yeast strain unable to grow in medium containing low potassium concentration to survive [CY 162, for example, see J. A. Anderson et al., Proc. Natl. Acad. Sci. USA 89, 3736-3740 (1992)]. Yeast vectors may contain an origin of replication from the endogenous 2 micron (2μ) yeast plasmid or an autonomously replicating sequence (ARS) which confer on the plasmid the ability to replicate at high copy number in the yeast cell, centromeric (CEN) sequences which limit the ability of the plasmid to replicate at only low copy number in the yeast cell, a promoter, DNA encoding the heterologous DNA sequences, sequences for polyadenylation and transcription termination, and a selectable marker gene. An exemplary plasmid is Yrp7, [Stinchcomb et al., Nature 282, 39 (1979); Kingsman et al., Gene 7, 141 (1979); Tschemper et al., Gene 10, 157 (1980)]. This plasmid contains the TRP1 gene, which provides a selectable marker for a mutant strain of yeast lacking the ability to grow in the absence tryptophan, for example ATCC No. 44076. The presence of the trp1 lesion in the yeast host cell genome then provides an effective environment for detecting transformation by growth in the absence of tryptophan.

Suitable promoting sequences in yeast vectors include the promoters for metallothionein (Yep52), 3-phosphoglycerate kinase [pPGKH, Hitzeman et al., J. Biol. Chem. 255, 2073 (1980)] or other glycolytic enzymes [pYSK153, Hess et al., J. Adv. Enzyme Reg. 7, 149 (1968)]; and Holland et al., Biochemistry 17, 4900 (1978)], such as enolase, glyceraldehyde-3-phosphate dehydrogenase, hexokinase, pyruvate decarboxylase, phosoph-fructokinase, glucose-6-phosphate isomerase, 3-phosphoglycerate mutase, pyruvate kinase, triosephosphate isomerase, phosphoglucost isomerase, and glucokinase. Suitable vectors and promoters for use in yeast expression are further described in R. Hitzeman et al., EPO Publn. No. 73,657. Other promoters, which have the additional advantage of transcription controlled by growth conditions, are the promoter regions for alcohol dehydrogenase 2 (pAD4M), isocytochrome C, acid phosphates, degradative enzymes associated with nitrogen metabolism, and the aforementioned metallothionein and glyceraldehyde-3-phosphate dehydrogenase, as well as enzymes responsible for maltose and galactose (pYES2) utilization. Finally, in constructing suitable expression plasmids, the termination sequences associated with these genes may also be ligated into the expression vector 3′ of the heterologous coding sequences to provide polyadenylation and termination of the mRNA.

In certain embodiments, the nucleic acid sequences of the invention are used to express proteins in a bacterial host. Protein expressed in bacteria can be used in raising antisera (both polyclonal and monoclonal) by standard methodology. Such antibodies are useful in immunohistochemical studies to determine the level of expression of the channel protein in various tissues and cell lines. The channel can be purified from bacterial cells if found in inclusion bodies, for example, by isolation of inclusion bodies by standard techniques, followed by electrophoresis in SDS-PAGE gels and isolation of the protein band from the gel. Alternately, the potassium channel proteins, or portions thereof, can be expressed as a fusion protein, e.g., with glutathione-s-transferase, or maltose binding protein, and then purified by isolation of the protein to which it is fused. In additional embodiments of the invention, the predicted amino acid sequence can be used to design synthetic peptides unique to the potassium channels as herein described, which peptides can then be used to raise antibodies to the channels.

The present invention further provides methods of identifying cells or tissues which express a member of the family of channel proteins presented herein. For example, a probe comprising a DNA sequence of hORK1, a fragment thereof, or a DNA sequence encoding another member of the hORK1 family of channel proteins can be used as a probe or amplification primer to detect cells which express a message homologous to the probe or primer. One skilled in the art can readily adapt currently available nucleic acid amplification or detection techniques so that it employs probes or primers based on the sequences encoding a member of this family.

The materials for use in these embodiments are ideally suited for the preparation of a kit. Specifically, a kit is provided, which is compartmentalized to receive in close confinement, one or more containers which comprises: (a) a first container comprising one or more probes or amplification primers based on the hORK sequence or any of the other sequences, or simply a fragment containing nucleic acids that encode ZXXZ₁Z₂Z₃GXG and Z₄X₁X₂X₃GX₄PX₅; and (b) one or more other containers comprising one or more of the following: a sample reservoir, wash reagents, reagents capable of detecting presence of bound probe from the first container, or reagents capable of amplifying sequences hybridizing to the amplification primers.

A compartmentalized kit includes any kit in which reagents are contained in separate containers. Such containers include small glass containers, plastic containers or strips of plastic or paper. Such containers allow one to efficiently transfer reagents from one compartment to another compartment such that the samples and reagents are not cross-contaminated and the agents or solutions of each container can be added in a quantitative fashion from one compartment to another. Such containers will include a container which will accept the test sample, a container which contains the probe or primers used in the assay, containers which contain wash reagents (such as phosphate buffered saline, Tris buffers, etc.), and containers which contain the reagents used to detect the bound probe or amplified product.

Types of detection reagents include labeled secondary probes, or in the alternative, if the primary probe is labeled, the enzymatic, or antibody binding reagents which are capable of reacting with the labeled probe. One skilled in the art will readily recognize that probes and amplification primers based on the sequence disclosed in the present invention can be readily incorporated into one of the established kit formats which are well known in the art.

The sequences of the present invention are also valuable for chromosome identification. The sequence may be specifically targeted to and hybridize with a particular location on an individual chromosome, for example, the human chromosome. Moreover, there is a current need for identifying particular sites on the chromosome. Few chromosome marking reagents based on actual sequence data (repeat polymorphisms) are presently available for marking chromosomal location. The mapping of DNA to chromosomes according to the present invention is an important first step in correlating those sequences with genes associated with disease, or tracking other possible disease pathways.

Briefly, sequences can be mapped to chromosomes by preparing PCR primers (preferably 15-25 bp) from the cDNA. Computer analysis of the cDNA is used to rapidly select primers that do not span more than one exon in the genomic DNA, thus complicating the amplification process. These primers are then used for PCR screening of somatic cell hybrids containing individual chromosomes. Only those hybrids containing the gene corresponding to the primer will yield an amplified fragment.

PCR mapping of somatic cell hybrids is a rapid procedure for assigning a particular DNA to a particular chromosome. Using the present invention with the same oligonucleotide primers, sublocalization can be achieved with panels of fragments from specific chromosomes or pools of large genomic clones in an analogous manner. Other mapping strategies that can similarly be used to map to its chromosome include in situ hybridization, prescreening with labeled flow-sorted chromosomes and preselection by hybridization to construct chromosome specific-cDNA libraries.

Fluorescence in situ hybridization (FISH) of a cDNA clones to a metaphase chromosomal spread can be used to provide a precise chromosomal location in one step. This technique can be used with cDNA as short as 500 or 600 bases; however, clones larger than 2,000 bp have a higher likelihood of binding to a unique chromosomal location with sufficient signal intensity for simple detection. FISH requires use of the large clones from which the cDNA was derived, and the longer the better. For example, 2,000 bp is good, 4,000 is better, and more than 4,000 is probably not necessary to get good results a reasonable percentage of the time.

Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

With current resolution of physical mapping and genetic mapping techniques, a cDNA precisely localized to a chromosomal region associated with the disease could be one of between 50 and 500 potential causative genes. (This assumes 1 megabase mapping resolution and one gene per 20 kb).

Comparison of affected and unaffected individuals generally involves first looking for structural alterations in the chromosomes, such as deletions or translocations that are visible from chromosome spreads or detectable using PCR based on that cDNA sequence. Ultimately, complete sequencing of genes from several individuals is required to confirm the presence of a mutation and to distinguish mutations from polymorphisms.

In yet another embodiment of the present invention, a yeast expression system is described, wherein yeast cells bear heterologous potassium channels. Cloning and expression of potassium channels from heterologous species such as those described herein are useful in the discovery of new pesticides, and animal and human therapeutics. Discovery of such compounds will necessarily require screening assays of high specificity and throughput. For example, new pesticides directed at potassium channels require high selectivity for insect channels and low activity against non-insect species. Screening assays utilizing yeast strains genetically modified to accommodate functional expression of heterologous potassium channels offer significant advantages in this area. In preferred embodiments, these channels expressed in heterologous yeast cells are dORK, RAK (as described below), Shal, Shaw, Eag, cORK, or hORK1. As noted above, transformed host cells of the present invention express the proteins or protein subunits coded for by the heterologous DNA sequences. When expressed, the potassium channel is located in the host cell membrane (i.e., physically positioned therein in proper orientation for both the stereoselective binding of ligands and passage of potassium ions). In other preferred screening embodiments of the present invention, the potassium channel is positioned within a cell membrane in such a manner as to allow it to function as a modulator of the flow of potassium ions into and out of the cell. To best regulate this activity, at least one pore-forming domain may be positioned proximal to a exterior portion of the cell membrane. Thus, in certain preferred screening embodiments of the present invention, a transformed yeast cell is presented, containing a heterologous DNA sequence which codes for a potassium channel, as herein presented, cloned into a suitable expression vector. Various other useful potassium channels may be utilized in the screening assay embodiments of the present invention, such as a delayed rectifier potassium channel referred to as “RAK or RATRAK” [Paulmichl et al., Proc. Natl. Acad. Sci, USA 88 7892-7895 (1991), reporting the cloning of this potassium channel from rat cardiac tissue.] RAK is capable of complementing the potassium-dependent phenotype of Saccharomyces cerevisiae strain CY162 on medium containing low potassium concentration.

Using the purified proteins, or polypeptide sequences of the invention, the present invention provides methods of obtaining and identifying agents capable of binding to or otherwise interacting with the potassium channels of the invention.

In detail, said method comprises:

(a) contacting a substance with a select member of the family of potassium channels or select channel peptides or proteins; and

(b) determining whether the substance interacts with said channel, peptide, or protein.

The screened substances in the above assay can be, but are not limited to, proteins, peptides, peptidomimetics, carbohydrates, vitamin derivatives, compounds, or other pharmaceutical agents or any mixtures thereof. The substances can be selected and screened at random or rationally selected or designed using protein modeling techniques. As used herein, a substance is said to be “rationally selected or designed” when the substance is chosen based on the configuration of the particular member of the claimed family of channel proteins. For example, one skilled in the art can readily adapt currently available procedures to generate peptides, pharmaceutical agents and the like capable of binding to a specific peptide sequence in order to generate rationally designed antipeptide peptides, for example see Hurby et al., “Application of Synthetic Peptides: Antisense Peptides,” In Synthetic Peptides, A User's Guide, W. H. Freeman, N.Y., 289-307 (1992), and Kaspczak et al., Biochemistry 28, 9230-8 (1989). Pharmaceutical agents and the like may be similarly generated using techniques known to the art.

The present invention further provides methods for modulating the expression of hORK, or a member of the hORK family of channel proteins. Specifically, anti-sense RNA expression is used to disrupt the translation of the mRNA encoding the hORK protein.

In detail, a cell is modified using routine procedures such that if expresses an antisense mRNA, an mRNA which is complementary to mRNA encoding the hORK family member. By constitutively or inducibly expressing the antisense RNA, the translation of the hORK family member mRNA can be regulated.

In certain preferred embodiments, the cloning of the members disclosed herein now makes possible the screening capability which enables the identification of agonists (potassium channel openers) and antagonists (potassium channel closers) of this family of channel proteins. The two-pore K⁺ channels described herein in humans can be used as targets for novel human therapeutics. The primary target for such therapeutic agents will be conditions related to alterations in the plasma membrane resting potential and/or the duration of the action potential in excitable cells. Potassium channels influence action waveforms and firing frequency of cells and therefore play a role in neuronal integration, muscle contraction, and hormone secretion in excitable cells. Potassium channels play the vital role of determining resting electrical membrane potential by setting membrane permeability to potassium ions in the cell. Inward conductance at membrane potentials below K⁺ equilibrium potential (Eₖ) prevents excessive hyperpolarization which may be caused by the electrogenic Na⁺ pump; the slight outward conductance of inward rectifier K⁺ channels at membrane potentials just above K⁺ equilibrium helps to keep the resting membrane potential close to Eₖ. Modulation of the conductance level of potassium channels changes the resting potential and alters the excitability of a cell; i.e. the activation of a particular type of inward rectifier K⁺ channel has been shown to cause hyperpolarization of the cardiac pacemaker cells and slows the heartbeat. Thus, modulation of potassium channels can occur when one provides to cells, agents capable of binding to the potassium channel proteins.

In the cardiovascular area, this class of potassium channels may be of use in the discovery of new agents for the treatment of atrial and ventricular arrhythmias, heart failure including associated arrhythmias and cardiac ischemia. The action of such agents would be effected through the modulation of the kinetics duration of the cardiac action potential.

Modulation of cardiac action potential by compounds that effect the behavior of potassium channels may be a useful treatment for serious heart conditions. The delayed rectifier potassium current in heart cells regulates the duration of the plateau of the cardiac action potential by countering the depolarizing, inward calcium current. Delayed rectifier potassium currents characteristically are activated upon depolarization from rest, display a sigmoidal or delayed onset, and have a nonlinear, or rectifying, current-voltage relationship. Several types of delayed potassium conductances have been identified in cardiac cells based on measured single-channel conductances. Heart-rate and contractility are regulated by second messenger modification of delayed rectifier potassium conductances, and species differences in the shape of the plateau may be influenced by the type and level of channel expression. Potassium channel openers may also function as smooth muscle relaxants, functioning as vasodilators, vasospasmolytics, and other smooth muscle spasmolytic. As vasodilators, these compounds have use as dilators of peripheral vasculature, coronary arteries, renal vasculature, cerebral vasculature, and mesenteric vasculature. As vasospasmolytics, these compounds have use in the treatment of coronary artery spasm, peripheral vascular spasm, cerebral vascular spasm and impotence. Other smooth muscle spasmolytics have use as bronchodilators, in the control of urinary bladder and gall bladder spasm, and in the control of esophageal, gastric, and intestinal smooth muscle spasm.

Potassium channel closers may function in the pancreas to enhance release of insulin, in the kidney as diuretics and renal epithelial anti-ischemic agents, as hypertensive agents for promoting vasoconstriction for use in hypotensive states as antiarrhythmic agents, and as agents for modifying cardiac muscle contractility.

Other uses for potassium channel agonists or antagonists include anticonfulsants, hair growth promoting agents, and agents effective in preventing or reducing skeletal muscle damage or fatigue.

Thus, in yet further preferred embodiments, methods of modulating cellular activity to provide theraeutic value are provided, by applying to a patient in need of such modulation, a substance capable of interacting with a potassium channel contained in the relevant cells of such patient and modulating the activity of same (a good example of which are cardiac cells, useful for cardiac modulation purposes). These aspects of the present invention relate to methods of modulating potassium channel activity, by affecting the ability of such channel to allow the flow of ions into, through, or out of a cellular membrane, and particularly when these ions are potassium ions. Certain substances whether biological or chemical in nature, may be applied to cell membranes having as an integral part of their structure, one or more potassium channels as presented herein, and particularly those comprising the amino acid sequences of SEQ ID NO: 2, SEQ ID NO: 4, SEQ ID NO: 36, SEQ ID NO: 46, or RAK, in an amount and for a time sufficient to affect the ability of the potassium channel to so regulate the flow of ions. Substances that are potassium channel blockers will inhibit the ability of the channel to regulate the flow of such ions. Substances that enhance such ability may be considered potassium channel “activators.”

Application of such substances may take the form of in vitro, ex vivo, or in vivo application, each in a formulation suitable to deliver the substance to the cell membrane and to sustain such delivery for a time sufficient to allow the substance to interact with the membrane. Appropriate formulations, concentrations of substances, application time, and other relevant parameters may be established by utilizing, inter alia, known assays for measuring ion channel current flow. Such compositions may comprise conventional delivery/carrier systems, e.g., liposome or phospholipid encapsulation, water or saline solutions, polymeric compositions, and the like. Another suitable endpoint one skilled in the art may utilize in optimizing these parameters, especially in the case of potassium channel blockers, is “cell death”. Such assays may be performed in vitro and extrapolated to in vivo conditions, or in some cases may be easily established directly in vivo the field of insecticides is instructive for this purpose. For example, by applying the substance directly to a test sample comprising the target insect pest (whole organism) and noting the appropriate parameters at which an acceptable percent of insect death is attained.

In certain other preferred embodiments, methods of selectively inhibiting insect pests are presented by applying to such insect pests a substance capable of selectively inhibiting the activity of a potassium channel contained in the cells of such insect, and comprising the amino acid sequence of SEQ ID NO:2, or a potassium channel biologically equivalent thereto. In the most preferred embodiments, the inhibitor will inhibit the activity of the aforementioned potassium channel without inhibition of other, non-homologous or otherwise non-equivalent potassium channels that may be present in species other than the targeted insect pest. It is envisioned that such other species may also be present at the site of application of the inhibitor, such as in a garden, crop, or other site wherein it is desired to control insect pests. In other preferred embodiments, methods of selectively inhibiting nematode pests are presented much in the same manner as discussed for control of insect pests, by applying to such pests a substance capable of selectively inhibiting the activity of a potassium channel contained in the cells of such pest, said potassium channel comprising the the amino acid sequence of SEQ ID NO:4, SEQ ID NO: 36, or potassium channels biologically equivalent thereto.

The present invention further provides methods for generating chimeric or transgenic animals 1) in which the animal contains one or more exogenously supplied genes which are expressed in the same temporal and spatial manner as a member of the family of channel proteins as presented herein, or 2) in which such member of this family of channel proteins has been deleted or overexpressed. Such chimeric and transgenic animals are useful in the further elucidation of the mechanisms of potassium channel function as well as their effect an animal physiology. These transgenic and chimeric animals are produced by utilization of techniques which are well known and well described in the technical literature, e.g., see U.S. Pat. No. 5,434,340 and scientific references cited therein discussing inter alia, the introduction of transgenes into the gumline of a non-human animal, herein incorporated by reference.

The following Examples are provided to further illustrate various aspects of the present invention. They are not to be construed as limiting the invention.

### EXAMPLE 1

Using the yeast expression technology and other teachings as set forth herein, the present inventors have isolated a single 2463 base pair cDNA fragment from an invertebrate source, designated Dm ORF1 [SEQ ID NO: 1], by complementation of the potassium-dependent phenotype of Saccharomyces cerevisiae strain CY162 (trk1Δ) on medium containing low potassium concentration [J. A Anderson et al., Proc. Natl. Acad. Sci USA 89 3736-3740 (1992)]. Dm ORFI contains a single long open reading frame encoding a protein of 618 amino acids [SEQ ID NO:2] that exhibits substantial amino acid identity to the pore-forming regions of other potassium channels. The DmORF1 contains structural features that distinguish it from other classes of potassium channels, including four hydrophobic domains capable of forming transmembrane helices (M1-M4) and two putative pore forming H5 domains found between transmembrane helices M1 and M2, and M3 and M4. Each pore forming H5 domain contains the Y/F-G dipeptide motif required for potassium selectivity [Heginbotham et al., Science 258 1152-1155, (1992)]. This work was expanded to clone a construct derived from C. elegans having a single open reading frame sufficient to encode a protein of 434 amino acids, designated pCORK.

A search of the GENBANK database for DNA and protein sequences similar to DmORF 1 revealed several cloned potassium channel sequences including a putative protein coding DNA sequence, F22b7.7, reported in the Caenorhabditis elegans genome sequencing project [Wilson et al., Nature 368, 32-38 (1994)]. The DNA sequence contained a single long open reading frame sufficient to encode a protein of 336 amino acids (predicted MW 38.5 kDa) with substantial homology to known potassium channel sequences.

Using the hybridization approach, a cDNA sequence designated CeORF 1 [SEQ ID NO: 38] was isolated by probing a Caenorhabditis elegans cDNA library with oligonucleotides designed using F22b7.7 DNA sequences [T. N. Davis and J. Thorner Meth. Enzymol. 139, 246-262 (1987)]. CeORF1 contains a single long open reading frame encoding a protein that exhibits substantial amino acid identity to pore-forming regions of other potassium channels. DNA sequences encoding a human putative two-pore potassium channel were cloned by polymerase chain reaction (PCR) from human brain cDNA. Degenerate oligonucleotides (5′ and 3′ oligo) used in the analysis were designed from a compilation of nucleotide sequences encoding the pore-forming domains of putative two pore potassium channels identified in a search of the GENBANK DNA sequence database.

CeORF1 and pCORK each contain structural features similar to DmORF1, including two putative pore forming H5 domains. Each pore forming H5 domain contains the Y/F-G dipeptide motif required for potassium selectivity [Heginbotham et al., Science 258, 1152-1155, (1992)]. These features form the basis of the designation of a new sub-family of potassium channels comprising DmORF1, CORK, CeORF1, hORK, and various other homologs. The particulars of this discovery is set forth in more detail below:

Recombinant Expression Library Screening.

Saccharomyces cerevisiae strain CY162 is described in Anderson, J. A. et al., Proc. Natl. Acad. Sci. USA 89, 3736-3740 (1992)]. Growth of bacterial strains and plasmid manipulations are performed by standard methods (Maniatis T., Molecular Cloning. Cold Spring Harbor Laboratory Press, 1982). Media conditions for growth of yeast, isolation of plasmid DNA from yeast, and DNA-mediated transformation of yeast strains are as described (Rose M. D., Methods in yeast genetics, Cold Spring Harbor Laboratory Press, 1990). A multifunctional expression library constructed in pYES2 and containing cDNA made from 3rd instar male Drosophila melanogaster mRNA is used as described [S. J. Elledge et al., Proc. Natl. Acad. Sci USA 88, 1731-1735 (1991)]. A multifunctional expression library constructed in pYES2 and containing cDNA made from mRNA obtained from all life stages of Caenorhabditis elegans is custom-made by Invitrogen Corporation.

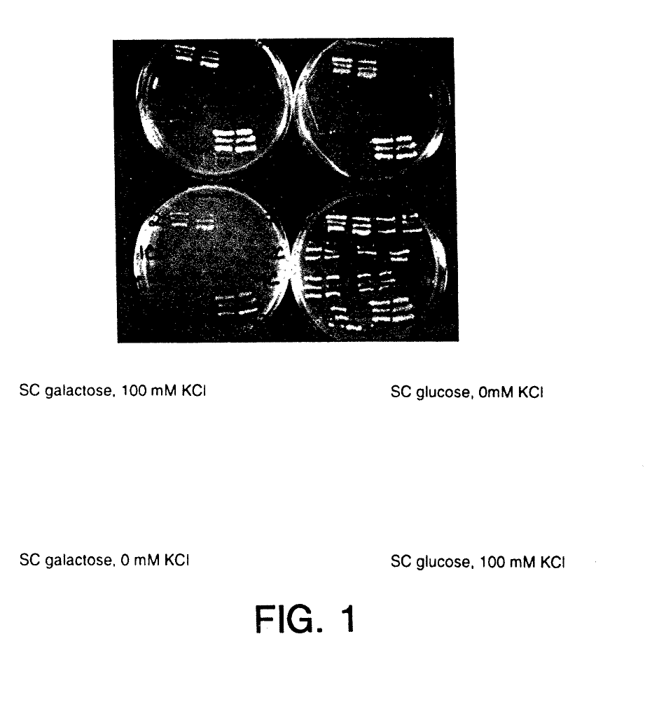

Isolation of expression plasmids encoding heterologous potassium channels. CY162 cells are transformed with plasmid DNA from each library to give 3×10⁶ transformants from each library on SCD-ura (synthetic complete dextrose (2%) medium containing all necessary nutritional supplements except uracil) containing 0.1 M KCl agar medium. Transformants are replica-plated to SCG-ura (synthetic complete galactose (2%) medium containing all necessary nutritional supplements except uracil) agar medium. Colonies that grow on this selective agar medium are transferred to SCG-ura agar medium to obtain single colonies clones and while reassaying suppression of the potassium-dependent phe-notype. Plasmid DNA is isolated from surviving colonies and used to transform CY162. Six individual transformant strains containing one plasmid, pDmORF1, that confers the potassium independent phenotype is cultured on SCD-ura and SCG-ura medium along with CY162 strains bearing pKAT1, which encodes a plant inward rectifier potassium channel that supports the growth of CY162 on selective medium (FIG. 1). The plasmid bearing strains exhibit potassium-independent growth on both dextrose and galactose containing medium. Growth on dextrose is likely due to basal level of transcription leading to sufficient potassium channel expression to support growth.

### EXAMPLE 2

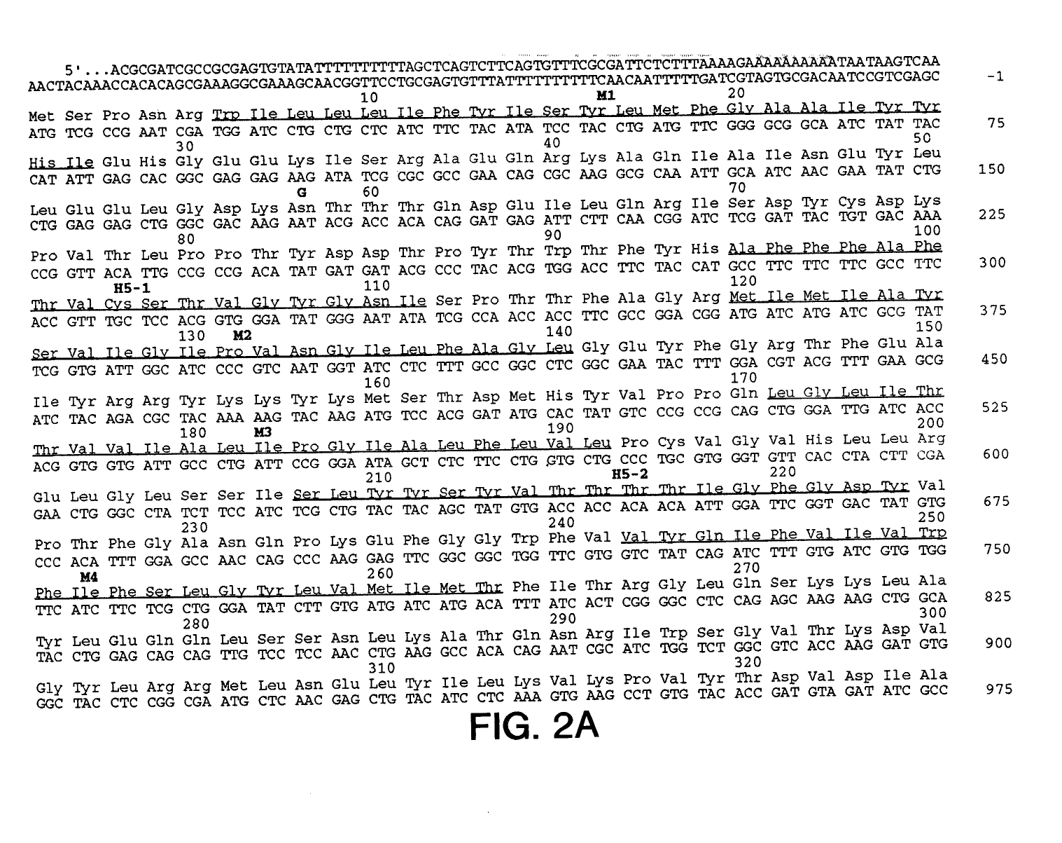

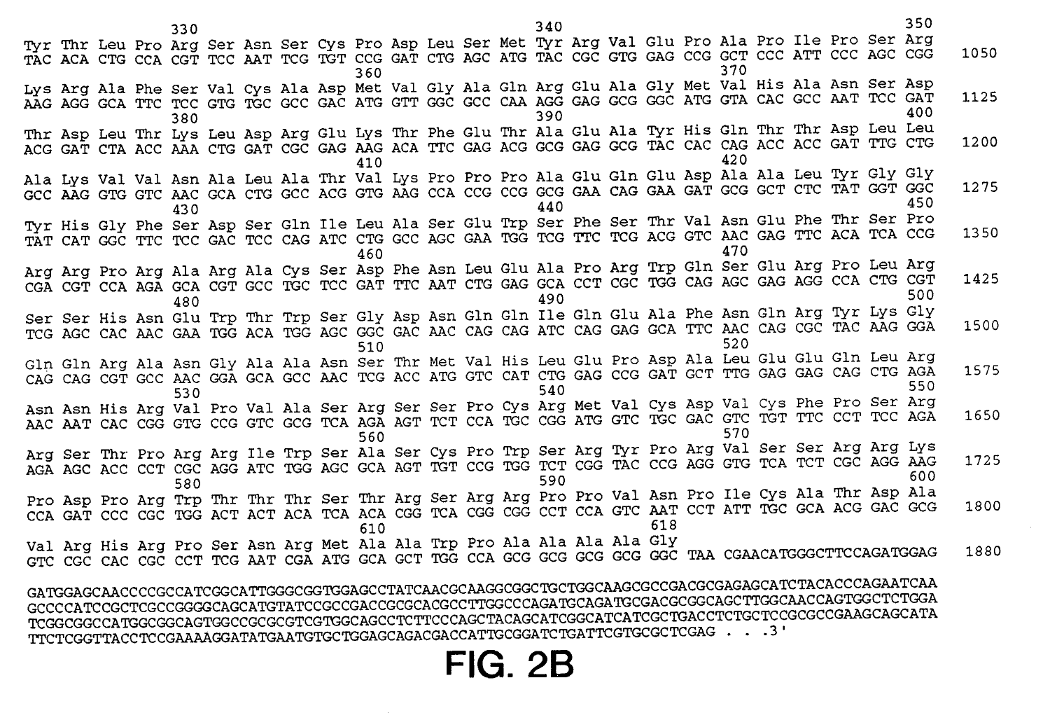

DNA sequence analysis of DmORF1. Plasmids that confer suppression of the potassium-dependent phenotype are subjected to automated DNA sequence analysis performed by high temperature cycle sequencing (Applied Biosystems). Geneworks DNA sequence analysis software (Intelligenetics) is used to align raw DNA sequence information and to identify open reading frames. The DNA sequence of the 2.4 kb insert in pDmORF1 is displayed in FIGS. 2A and 2B [SEQ ID NO: 1]. The 5′ untranslated sequences of the cDNA contain long poly A and poly T tracts not likely to be found in protein coding regions. The first ATG proximal to the 5′ end is present in a consensus Drosophila melanogaster translational initiation site [D. R. Cavener, Nucleic Acids Res., 15, 1353-1361 (1987)], consistent with the designation of this site as the translational start site. A single long open reading frame sufficient to encode a protein of 618 amino acids (predicted MW 68 kDa) is encoded in pDmORF1. A consensus polyadenylation site, AATCAA, occurs at position 2093-2098 in 3′ untranslated sequences. The DmORF1 contains structural features that distinguish it from other classes of potassium channels, including four hydrophobic domains capable of forming transmembrane helices (M1-M4) and two pore forming H5 domains found between transmembrane helices M1 and M2, and M3 and M4. Each pore forming H5 domain contains the Y/F-G dipeptide motif required for potassium selectivity [Heginbotham et al., Science 258, 1152-1155, (1992)].

### EXAMPLE 3

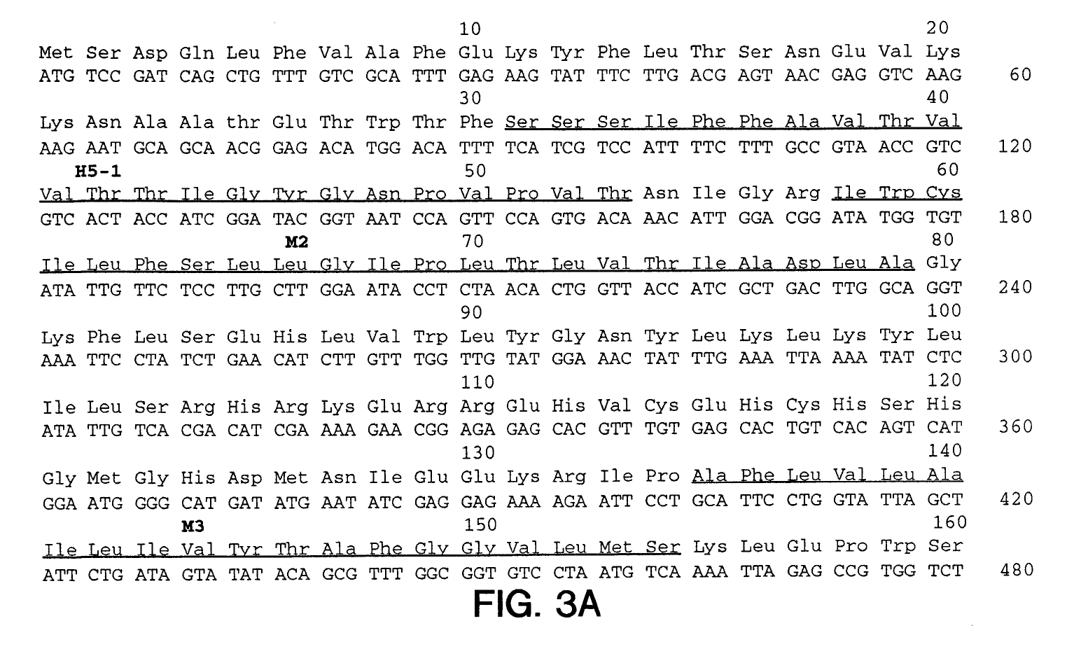

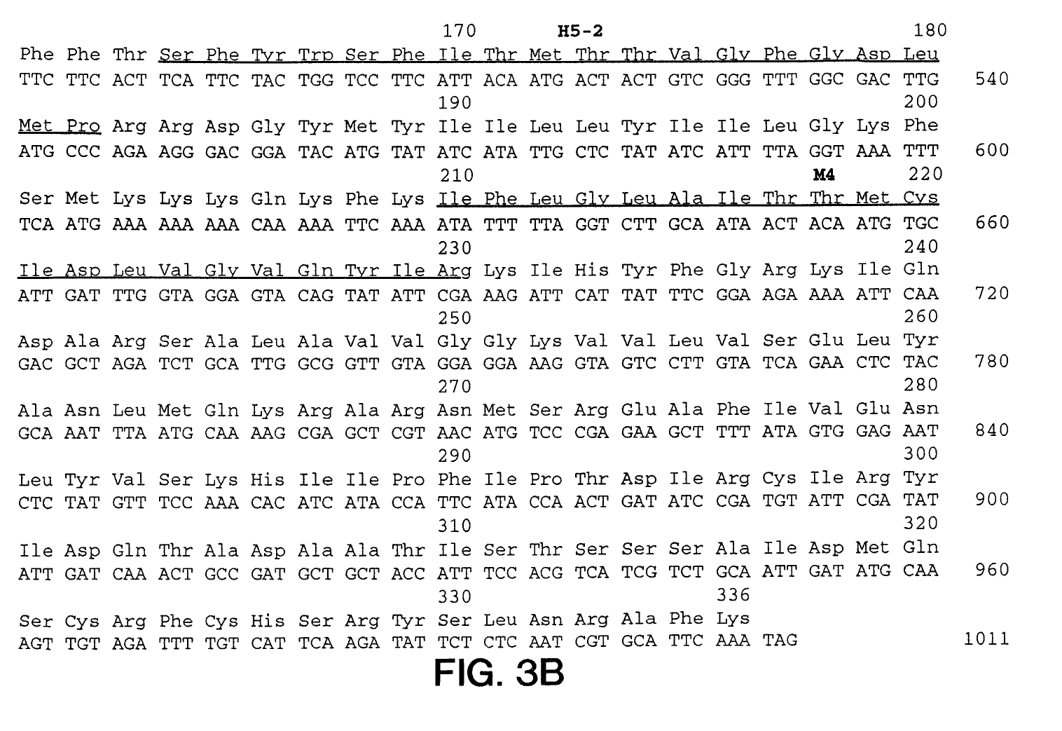

Identification of Caenorhabditis elegans sequences homologous to DmORF1. A search of the GENBANK database protein sequences similar to DmORF1 reveals significant matches with several known potassium channel sequences. The closest match is to a putative protein coding DNA sequence, F22b7.7, reported in the Caenorhabditis elegans genome sequencing project [Wilson et al., Nature 368, 32-38 (1994)]. The DNA sequence and predicted amino acid sequence assembled from putative exons recognized by a GENBANK exon identification algorithm is displayed in FIGS. 3A and 3B [SEQ ID NOS:3 and 4]. The DNA sequence contains a single long open reading frame sufficient to encode a protein of 336 amino acids (predicted MW 38.5 kDa) with substantial homology to known potassium channel sequences. The F22b7.7 sequence contains structural features that distinguish it from other classes of potassium channels, including three of four hydrophobic domains capable of forming transmembrane helices (M1-M4) identified in DmORF1 and two pore forming H5 domains found between transmembrane helices a predicted M1 and M2, and M3 and M4. Each pore forming H5 domain contains the Y/F-G dipeptide motif required for potassium selectivity [Heginbotham et al, Science 258 1152-1155, (1992)]. The lack of an amino terminal transmembrane domain homologous to DmORF1 M1 in the F22b7.7 sequence may be due to failure of the search algorithm to identify exon(s) encoding the amino terminus. Alternatively, an amino terminal coding sequence may be added by trans-splicing, which occurs frequently in Caenorhabditis elegans.

### EXAMPLE 4

Cloning and DNA sequence analysis of CeORF1. Oligonucleotides corresponding to DNA sequences encoding the two pore forming domains of F22b7.7 are synthesized using an Applied Biosystems DNA synthesizer.

F22b7.7-H2-1: 5′TCCATTTTCTTTGCCGTAACCGTCGTCACTACCATCGGATACGGTAATCCA [SEQ ID NO:5]. F22b7.7-H2-2: 5′TCATTCTACTGGTCCTTCATTACAATGACTACTGTCGGGTTTGGCGACTTG [SEQ ID NO:6]. The oligos were labelled at their 5′ ends with p using a 5′-end labelling kit according to manufacturers instructions (New England Nuclear). The labelled oligos are pooled and used to screen 6×10⁵ plaques from a λZAP-Caenorhabditis elegans cDNA library (obtained from Clontech) by published methods [T. N. Davis and J. Thorner Meth. Enzymol. 139, 246-262 (1987)]. Hybridization is at 42° C. for 16 hours. Positive clones are plaque-purified by twice repeating the hybridization screening process. Plasmid DNAs, excised from phage DNA according to manufacturers instructions, are subjected to automated DNA sequence analysis performed by high temperature cycle sequencing (Applied Biosystems). Geneworks DNA sequence analysis software (Intelligenetics) is used to align raw DNA sequence data and to identify open reading frames.

### EXAMPLE 5

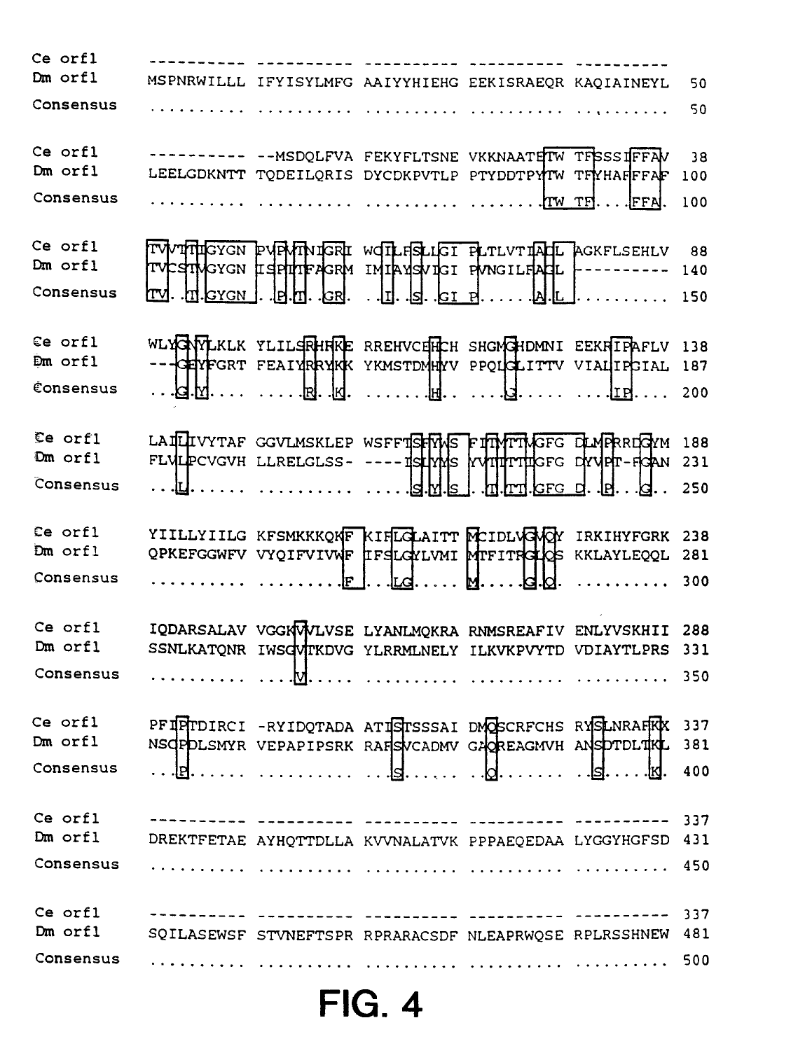

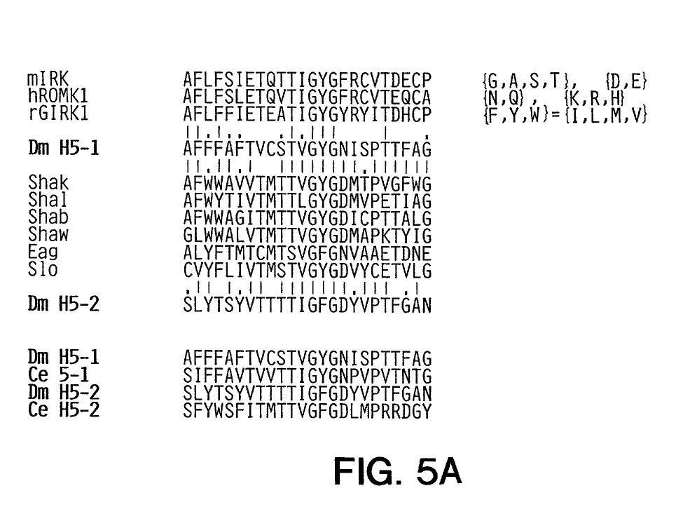

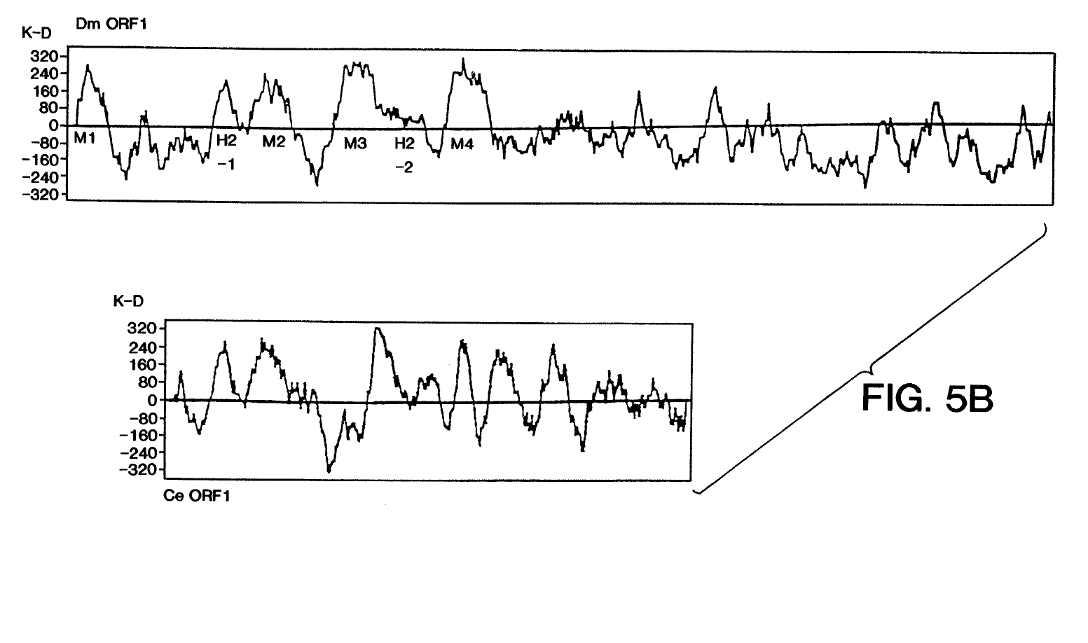

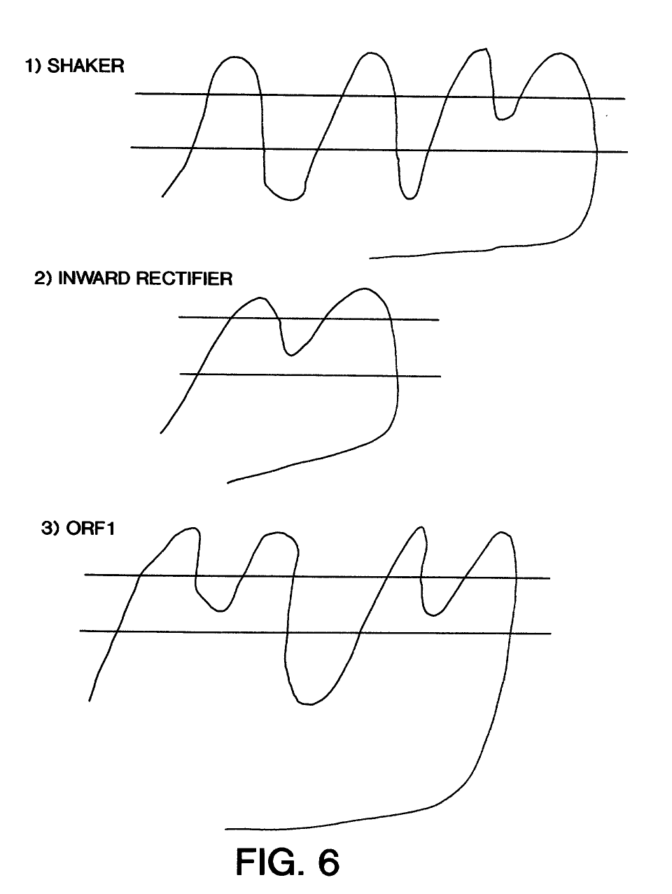

Comparison of the putative proteins encoded by DmORF1 and F22b7.7. Predicted amino acid sequences of DmORF1 and F22b7.7 are aligned and displayed in FIG. 4 [SEQ ID NOS:37 and 38]. Only limited overall amino acid homology is exhibited by these two proteins with regions of greatest homology existing in the pore forming H2-1 and H2-2 domains. FIG. 5A shows a comparison of the pore forming domains of DmORF 1 and F22b7.7 with those of the known Drosophila melanogaster potassium channel and inward rectifier sequences [SEQ ID NOS:7 through 21]. Amino acid identities greater than 50% are observed with all potassium channel sequences. FIG. 5B shows hydropathy plot analysis of DmORF1 and F22b7.7. The two proteins, which show remarkable topological similiarity through their length, are predicted to be composed of four membrane-spanning hydrophobic domains (M1-M4), and two pore forming H2 domains. These data suggest the predicted topology shown in FIG. 6. Both proteins are predicted to span the membrane four times with amino and carboxyl termini residing within the cell. This topology places the single amino-terminal asparagine-linked glycosylation site and H2 domains on the cell exterior permitting permeation of the membrane by the pore forming domains from the outside, an absolute requirement for the formation of a functional potassium channel.

### EXAMPLE 6

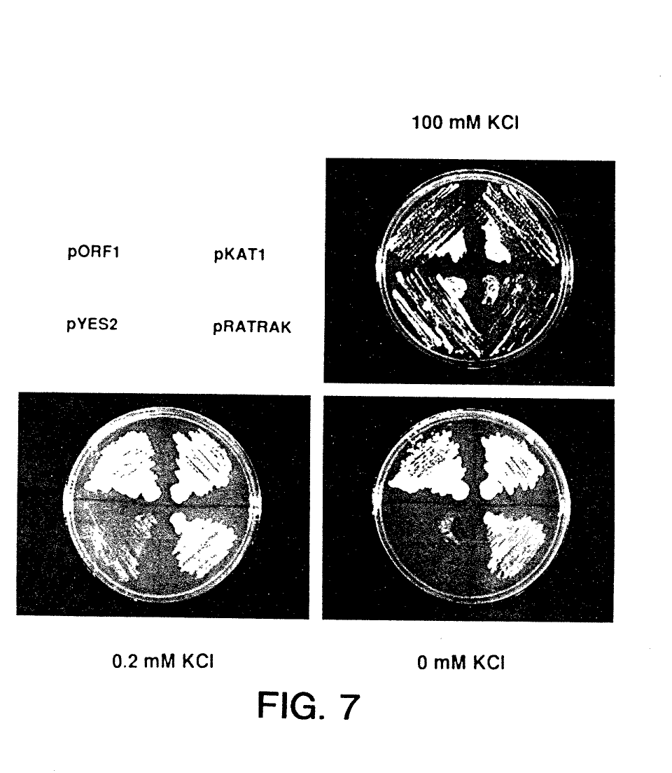

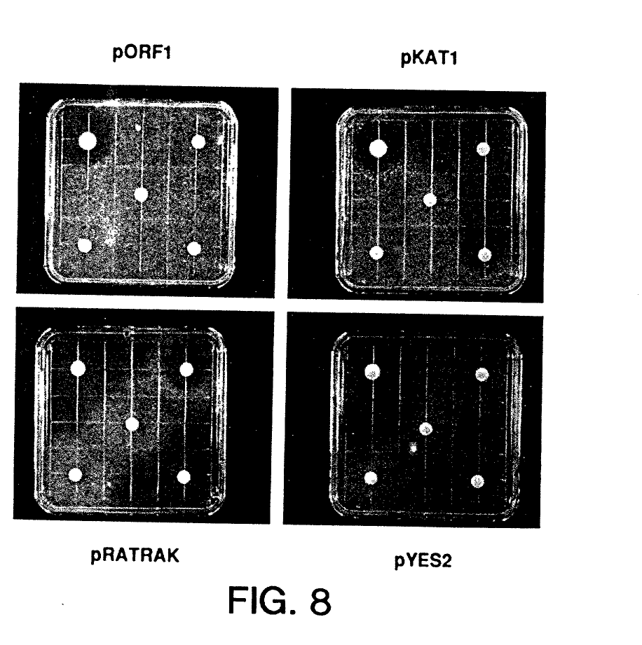

Functional expression of a rat atrial delayed rectifier potassium channel in yeast. CY162 transformants containing plasmids pKAT1, which encodes a plant inward rectifier potassium channel, pRATRAK, which encodes a rat atrial delayed rectifier potassium channel, pDmORF1, and control plasmid pYES are cultured on arginine-phosphate-dextrose agar medium lacking ura medium [A. Rodriguez-Navarro and J. Ramos, J. Bacteriol. 159, 940-945, (1984)] containing various KCl concentrations (FIG. 7). Strains containing pKAT1, pRATRAK, and pDmORF1 all support the growth of CY162 on medium containing a low concentration of potassium, while pYES2 containing CY162 cells only grow on medium containing a high potassium concentration, indicating that heterologous potassium channels of several different types function to provide high affinity potassium uptake.

pRATRAK is constructed by modifying the protein-coding sequences of RATRAK to add 5′ HindIII and 3′ XbaI sites using PCR. In addition, four A residues are added to the sequences immediately 5′ proximal to the initiator ATG to provide a good yeast translational initiation site. The modified fragment is cloned into the HindIII and XbaI sites in the yeast expression vector pYES2 (Invitrogen), forming pRATRAK.

### EXAMPLE 7

Bioassay of Functional Expression of Heterologous Potassium Channels.

Yeast strains dependent on heterologous potassium channels for growth should be sensitive to non-specific potassium channel blocking compounds. To test the potassium channel blocking properties of several compounds, a convenient agar plate bioassay is employed. Strains containing pKAT1, pRATRAK, pDmORF1, and pYES2 are plated in arginine-phosphate-dextrose agar medium lacking ura and containing various amounts of potassium chloride. Arginine-phosphate-dextrose medium is used to avoid interference from potassium and ammonium ions present in standard synthetic yeast culture medium. Sterile filter disks were placed on the surface of the agar and saturated with potassium channel blocking ions CsCl, BaCl₂, and TEA. The growth of heterologous potassium channel containing strains is inhibited by potassium channel blocking ions, in a channel dependent manner. DmORF 1-dependent growth is blocked by BaCl₂ but not by CsCl or TEA. KAT-dependent growth is blocked by BaCl₂, CsCl and TEA. RATRAK-dependent growth is blocked by BaCl₂, CsCl and TEA to a much greater extent than pKAT1, reflecting in part a slower growth rate of pRATRAK-containing cells. These observations confirm that these channels support the growth of the mutant yeast cells and demonstrate the efficacy of the yeast bioassay for screening for compounds that block potassium channel function. The control pYES-containing strain grows only around applied KCl and RbCl, a congener of KCl.

### EXAMPLE 8

Identification of Compounds that Alter Potassium Channel Activity.

Yeast strains made capable of growing on medium containing low potassium concentration by expression of heterologous potassium channels are used to screen libraries of chemical compounds of diverse structure for those that interfere with channel function. CY162 cells containing pKAT1, pRATRAK, pDmORF1, pCeORF1, and pYES2-TRK1 (10⁴/ml) are plated in 200 ml of arginine-phosphate-dextrose agar medium lacking ura and containing 0.2 mM potassium chloride in 500 cm² plates. The CY162 cells bearing pYES2-TRK1 are included in the assay as a control to identify compounds that have non-specific effects on the yeast strain and are therefore not specifically active against the heterologous potassium channels. Samples of chemical compounds of diverse structure (2 μl of 10 mg/ml solution in DMSO) are applied to the surface of the hardened agar medium in a 24×24 array. The plates are incubated for 2 days at 30° C. during which time the applied compounds radially diffuse into the agar medium. The effects of applied compounds on strains bearing heterologous potassium channel genes are compared to the pYES2-TRK1 bearing strain. Compounds that cause a zone of growth inhibition around the point of application that is larger on plates containing cells bearing the heterologous potassium channels than that observed around the pYES2-TRK1 bearing strains are considered selective potassium channel blockers. Compounds that induce a zone of enhanced growth around the point of application that is larger on plates containing cells bearing the heterologous potassium channels than that observed around the pYES2-TRK1 bearing strains are considered selective potassium channel openers.

### EXAMPLE 9

DmORF1-Induced Currents in X. laevis oocytes Assayed by Two-Electrode Voltage Clamp.

DNA sequence analysis of the pDmORF1 insert strongly suggest that the protein encoded by the single long ORF possesses properties in common with known potassium channels. To test this hypothesis, the electrophysiological properties of the putative potassium channel encoded by DmORF1 was examined by expression in X. laevis oocytes. Currents were measured by two-electrode whole-cell voltage clamp. DNA sequences encoding the open reading frame of DmORF1 were amplified by polymerase chain reaction (PCR) using the following oligonucleotides: MPO23: ATAAAGCTTAAAAATGTCGCCGAATCGATGGAT [SEQ ID NO:22] MPO24: AGCTCTAGACCTCCATCTGGAAGCCCATGT [SEQ ID NO:23] The full length PCR product was cloned into corresponding sites in pSP64 poly A (Promega), forming pMP147. Template DNA was linearized with EcoRI and RNA transcribed using the Message Machine (Ambion) in vitro transcription kit according to manufacturers instructions. A sample of the RNA was resolved in a MOPS-acetate-formaldehyde agarose gel and RNA content was estimated by ethidium bromide staining. The remainder was stored on dry ice. X. laevis oocytes were isolated and injected with 50 nl of sterile TE containing 5-20 ng transcript according to published procedures. After three days, whole oocyte currents were recorded using a two-electrode voltage clamp. Electrodes contained 3M KCl and had resistances of 0.3-1.0 MW. Recordings were performed with constant perfusion at room temperature in the presence of either low (10 mM) or high (90 mM) potassium chloride. Two electrode voltage clamp analysis of the DmORF 1 gene product expressed in X laevis oocytes demonstrates properties of a voltage- and potassium-dependent potassium channel. At low potassium concentrations, DmORF 1 exhibited outward current at depolarizing potentials. At high potassium concentration, DmORF1 exhibits both inward and outward currents. The DmORF1 channel displays a high preference for potassium and shows cation selectivity in the rank order K>Rb>NH₄>Cs>Na>Li. Potassium currents were greatly attenuated by BaCi₂.

### EXAMPLE 10

Developmental Regulation of DmORF1 Expression in D. melanogaster Determined by Northern Blotting Analysis.

Isolation of pDmORF1 from a D. melanogaster expression library strongly suggests that the insert contained within originated in mRNA from that species. Detailed understanding of the developmental regulation of DmORF1 expression should aid in determining strategies for use of DmORF 1 as a target for novel insecticides. To characterize DmORF1 expression, northern blotting analysis of poly A RNA from various stages of the D. melanogaster life cycle was carried out.

D. melanogaster poly A+ RNA from embryo, larvae and adult forms (Invitrogen, 5 mg) was resolved in a MOPS-acetate-formaldehyde agarose gel according to standard procedures. The gel was stained with ethidium bromide and photographed to mark the positions of 18 S and 28 S ribosomal RNAs used as molecular weight markers. RNA was transferred by capillary action to nitrocellulose with 10×SSPE. The blot was air-dried, baked for one hour at 80° C., and prehybridized in 4×SSPE, 1% SDS, 2× Denhardt's, 0.1% single stranded DNA at 68° C. for 2 hours.

A 2.4 kb XhoI fragment of DmORF 1 was isolated from pDmORF 1 and labeled with α-³²P dCTP using the Ready-to-Go kit (Pharmacia) according to manufacturers instructions. The probe was denatured by heating to 100° C. for 5 minutes followed by quenching in an ice water bath. The probe was added to the prehybridization solution and hybridization continued for 24 hours at 68° C.

The blot was washed briefly with 2×SSPE, 0.1% SDS at room temperature followed by 0.5×SSPE, 0.1% SDS at 65° C. for 2 hours. The blot was air-dried and exposed to Reflection X-ray film (NEN) using an intensifying screen at −70° C. for 48 hours.

Northern blotting analysis indicates that the DmORF1 probe hybridizes to an mRNA species of approximately 2.8 kb isolated from D. melanogaster embryo, larvae, and adult forms. The length of the DmORF1 mRNA corresponds well with the length of the predicted ORF. Thus, the DmORF is expressed at all developmental stages in the life cycle of D. melanogaster.

### EXAMPLE 11

Expression of the DmORF1 Gene Product in vitro.

DNA sequence analysis of the pDmORF1 insert reveals a single long ORF with conserved amino acid sequence domains in common with known potassium channels. The DNA sequence predicts an ORF sufficient to encode a protein of 618 amino acid in length. The DmORF1 polypeptide contains four segments of at least 20 hydrophobic amino acids in length suggesting that the segments span the plasma membrane. In addition, the DmORF 1 protein sequence contains a putative N-linked glycosylation site (Asn-Thr-Thr) at amino acids 58-60. To confirm that a protein of the predicted size of DmORF is expressed from the insert in pDmORF1 and to test the proposition that DmORF 1 is glycosylated, pDmORF 1 was used as template to drive coupled in vitro transcription/translation.

Plasmid pMP147 was used as template to produce ³⁵S-labeled DmORF1 gene product in vitro using a TnT coupled transcription-translation kit (Promega) according to manufacturers instructions. Glycosylation of the nascent DmORF1 poly-peptide was accomplished by addition of canine pancreatic microsomes (Promega) to the transcription-translation reaction. Samples of glycosylated DmORF protein were treated with endoglycosidase H to remove added carbohydrate moieties. Aliquots were precipitated with TCA and collected on GF/C filters, washed with ethanol, dried and counted. Equivalent cpm's were resolved by SDS-PAGE. The gel was impregnated with soluble fluor Amplify (Amersham) and dried onto Whatman 3MM paper. The dried gel was exposed to Reflection X-ray film at room temperature.

Translation of the DmORF 1 gene product in vitro produced a polypeptide of 68 kDa, consistent with the predicted molecular weight of the ORF. Translation of DmORF 1 in the presence of canine pancreatic microsomes results in synthesis of a protein with reduced electrophoretic mobility, consistent with glycosylation of the nascent polypeptide. Treatment of glycosylated DmORF with EndoH increased its relative mobility as expected upon removal of carbohydrate moieties. Thus, the pDmORF 1 insert is capable of directing the expression of a glycoprotein with the expected molecular weight. EndoH treatment removes carbohydrate residues consistent with the sugar added through N-linked glycosylation.

### EXAMPLE 12

High-Affinity K⁺ Uptake and Selectivity of DmORF1 Expressed in Yeast.

Expression of DmORF permits CY162 cells to grow on medium containing a low concentration of potassium, implying that DmORF1 supplies high affinity potassium uptake capacity. To characterize the potassium uptake properties of CY162 cells containing DmORF1, Rb uptake studies were performed. Examination of the uptake of this potassium congener revealed important aspects of potassium uptake by DmORF1.

Yeast strains containing heterologous potassium-expression plasmids CY162-DmORF1, CY162-pKAT and the control strain CY162-pYES2 (Clontech) were cultured overnight in SC Gal-ura containing 0.1 M KCl. The cells were harvested, washed with sterile doubled distilled water and starved for K⁺ for 6 hours in Ca-MES buffer. Cells were washed again and distributed to culture tubes (10⁸ cells/tube) containing 86RbCl in Ca-MES buffer. The tubes were incubated at room temperature, samples filtered at various time intervals and counted. 86Rb uptake into cells was displayed.

The high-affinity potassium uptake capacity encoded by DmORF1 permits high-affinity uptake of the potassium congener, 86Rb, as well. Barium inhibited 86Rb uptake. No high affinity 86Rb uptake is observed in control CY162-pYES2 cells and 86Rb uptake into CY162-pKAT cells is consistent with its published properties.

### EXAMPLE 13

Expression of Drosophila melanogaster Potassium Channels in Yeast.

Voltage-gated potassium channel diversity in the fruitfly Drosophila melanogaster is encoded in large part by six genes, Shaker, Shab, Shal, Shaw, Eag, and Slo. Expression of these potassium channels in yeast will permit their introduction into screening assays for novel insecticidal compounds and facilitate characterization of their ion channel properties and sensitivity to compounds with activating and inhibitory properties.

DNA sequences encoding Drosophila melanogaster potassium channels were amplified by PCR using synthetic oligonucleotides that add 5′ HindIII or Kpn I, sites and 3′ XbaI, SphI, or XhoI sites:

| | |
|---|---|
| Shaker 5′: | |
| AAAAAGCTTAAAATGGCACACATCACG | [SEQ ID NO:24] |
| Shaker 3′: | |
| AAACTCGAGTCATACCTGTGGACT | [SEQ ID NO:25] |
| Shab 5′: | |
| AAAAAGCTTAAAATGGTCGGGCAATTG | [SEQ ID NO:26] |
| Shab 3′: | |
| AAAAGCATGCTCATCTGGATGGGCA | [SEQ ID NO:27] |
| Shal 5′: | |
| AAAAAGCTTAAAATGGCCTCGGTCGCC | [SEQ ID NO:28] |
| Shal 3′: | |
| TTTTCTAGACTACATCGTTGTCTT | [SEQ ID NO:29] |
| Shaw 5′: | |
| AAAAAGCTTAAAATGAATCTGATCAAC | [SEQ ID NO:30] |
| Shaw 3′: | |
| AAATCTAGATTAGTCGAAACTGAA | [SEQ ID NO:31] |
| Eag 5′: | |
| AAAAAGCTTAAAATGCCTGGCGGA | [SEQ ID NO:32] |
| Eag 3′: | |
| AAATCTAGAGGCTACAGGAAGTCC | [SEQ ID NO:33] |
| Slo 5′: | |
| GGGGGTACCAAAATGTCGGGGTGTGAT | [SEQ ID NO:34] |
| Slo 3′: | |
| TTTTTCTAGATCAAGAGTTATCATC | [SEQ ID NO:35] |

Plasmids used as templates for the PCR reactions were: pBSc-DShakerH37, pBSc-dShab11, pBSc-dShal2+(A)₃₆, pBScMXT-dShaw [A. Wei et al., Science 248, 599-603 (1990), provided by L. Salkoff], pBScMXT-slo,v4 [Atkinson et al., Science 253 551-555, (1991), provided by L. Salkoff], and pBIMCH20 Eag [CH20] [Warmke et al., Science 252, 1560-1564 (1991), Bruggemann et al., Nature 365, 445-448 (1993), provided by B. Ganetzky].

Amplified fragments were digested with the appropriate restriction endonucleases, purified using GeneClean (Bio 101), and ligated into corresponding sites in pYES2 (Invitrogen). CY162 cells were transformed with assembled Drosophila melanogaster potassium channel expression plasmids by the LiCl method and plated on SCD-ura containing 0.1M KCl agar medium. Selected transformants were tested for growth on arginine-phosphate-galactose (2%)/sucrose (0.2%)-ura agar medium containing 1-5 mM KCl. CY162 cells containing pKAT1 or pDmORF1 were cultured as positive controls and CY162 cells containing pYES2 were grown to provide a negative control.

CY162 cells bearing Drosophila melanogaster potassium channel expression plasmids survive under conditions in which growth is dependent on functional potassium channel expression. At potassium ion concentrations between 1-3 mM, negative control CY162 cells containing pYES2 grow poorly. Expression of the Drosophila melanogaster potassium channels Shal, Shaw and Eag substantially improve growth of CY162. These results are consistent with the Drosophila melanogaster potassium channels providing high-affinity potassium uptake capacity. This capacity is apparently sufficient to replace the native high-affinity potassium transport capacity encoded by TRK1 which is lacking in CY162 (trk1 trk2) cells.

### EXAMPLE 14

Cloning of a Novel C. elegans Sequence with Homology to Potassium Channels.

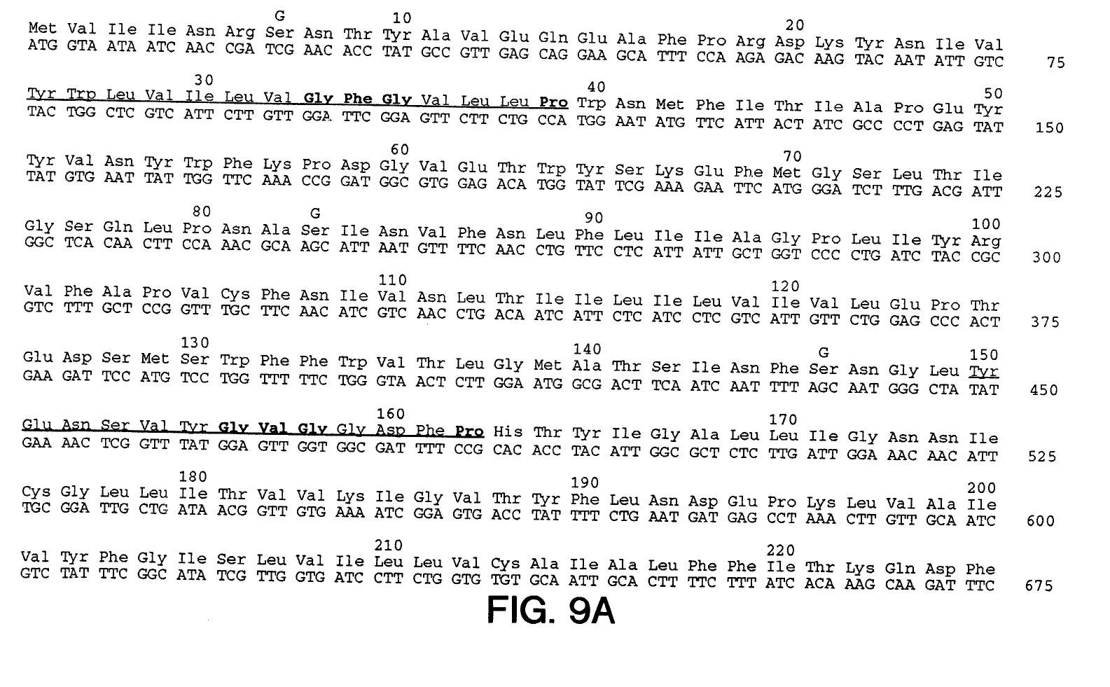

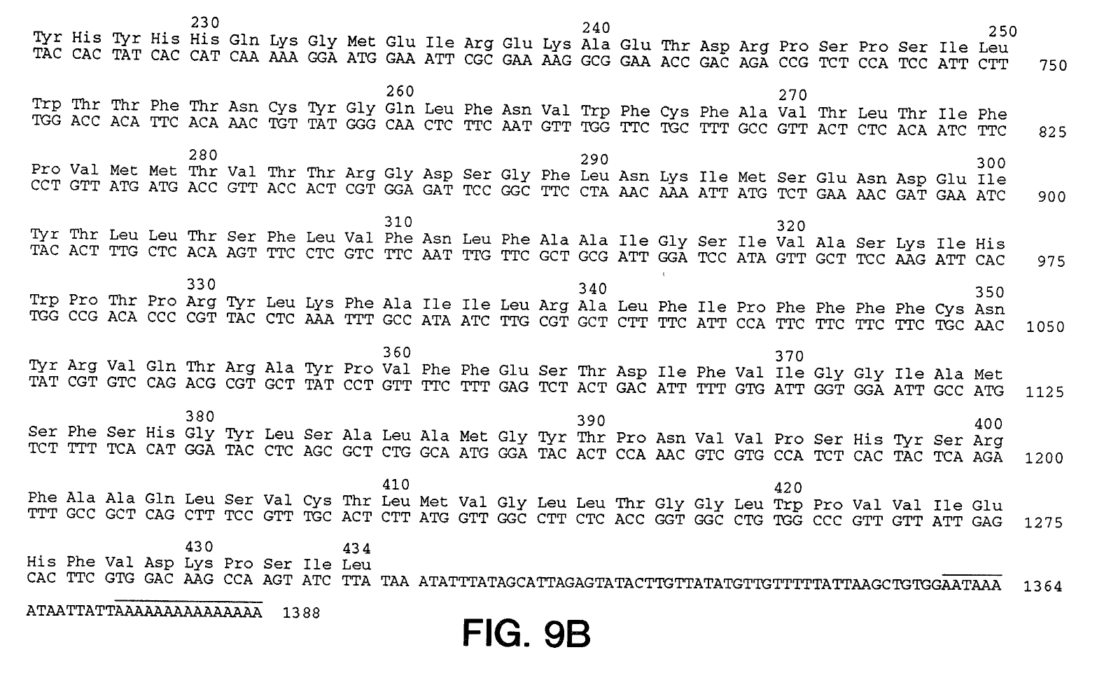

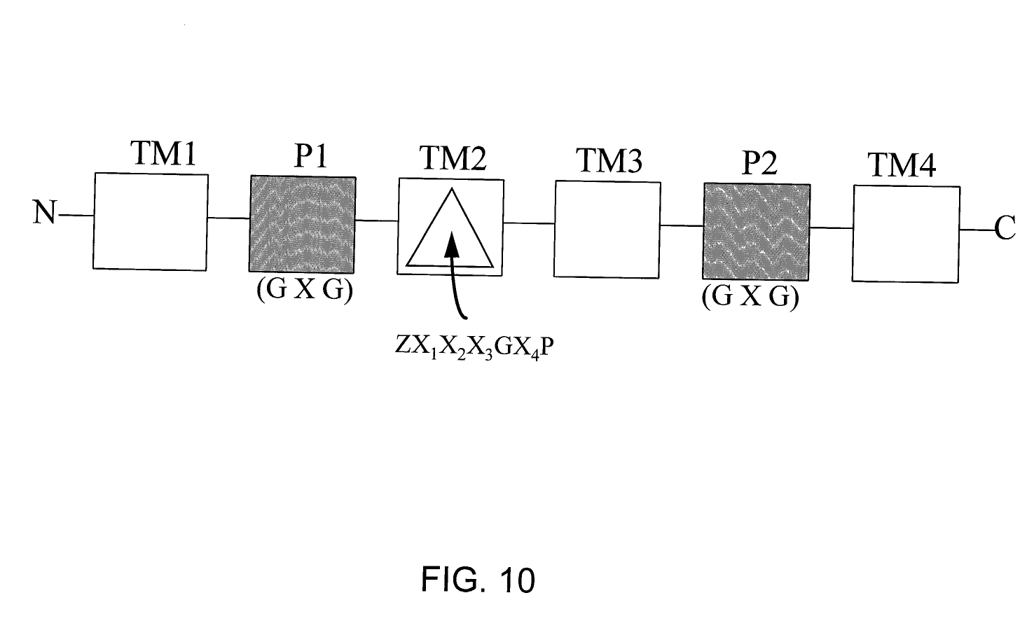

In order to expand the applicability of this technology to discover compounds with novel anhelmenthic activity, CY162 cells were transformed with a pYES2-based yeast expression library constructed using cDNA synthesized from C. elegans mRNA (Invitrogen). Plasmid DNA isolated from yeast cells that survived the selection scheme described in EXAMPLE 1 were subjected to automated DNA sequence analysis performed by high temperature cycle sequencing (Applied Biosystems). Geneworks DNA sequence analysis software (Intelligenetics) is used to align raw DNA sequence information and to identify open reading frames. The DNA sequence of the 1.4 kb insert in pCORK is displayed in FIGS. 9A and 9B [SEQ ID NO:36]. The 5′ untranslated sequences of the cDNA are present in this construct. A single long open reading frame sufficient to encode a protein of 434 amino acids (predicted MW 48 kDa) is predicted in pCORK. A consensus polyadenylation site, AATAAA, occurs at position 1359-1364 in 3′ untranslated sequences and is followed by a tract of 15 consecutive A residues. The CORK ORF contains structural features that resemble pore forming H5 domains found in potassium channels. Two putative pore forming H5 domains (residues 76-39 and 150-162) contain the G-Y/F-G tripeptide motif required for potassium selectivity [Heginbotham et al., Science 258, 1152-1155, (1992)].

### EXAMPLE 15

Cloning of the Human Two-Pore Potassium Channel Sequence: hORK1.

### Materials and Methods

DNA sequences encoding a human putative two-pore potassium channel were cloned by polymerase chain reaction (PCR) from human brain cDNA. Degenerate oligonucleotides (5′ and 3′ oligo) used in the analysis were designed from a compilation of nucleotide sequences encoding the pore-forming domains of putative two pore potassium channels identified in a search of the GENBANK DNA sequence database.

Oligos used in degenerate PCR cloning approach

| | | |
|---|---|---|
| 5′ oligo: | 5′ TIG GAT (AT)(CT)G G(AT)G A(CT)(AT) T | [SEQ ID NO:39] |
| 3′ oligo: | 5′ (AG)TC (AT)CC (AG)(AT)A (ACT)CC (AGT)A(CT) (AGT)GT | [SEQ ID NO:40] |

Clontech QUICK-Clone human brain cDNA was used as template (1 ng cDNA in 20 μl reaction) in a reaction mixture containing 1.25 U AmpliTaq DNA Polymerase (Perkin-Elmer), 1 μM primers, 200 μM dNTPs. PCR was carried out by standard procedures using the cycles given below in a Perkin-Elmer 9600 thermocycler.

| | | |
|---|---|---|
| PCR: | 94° 2′ | 1 cycle |
| | 94° 30″ | |
| | 48° 30″ | 35 cycles |
| | 60″ ramp to 72° | |
| | 72° 30″ | |
| | 72° 10′ | |

The resulting PCR fragments were cloned into the Invitrogen TA cloning kit according to manufacturers instructions. The cloned DNA fragments were sequenced with ABI Ready Reaction DyeDeoxy Terminator Cycle Sequencing Kit on the ABI373 Automated DNA sequencer according to manufacturers instructions. One fragment contained a 339 base pair (bp) open reading frame (ORF) with two consensus pore forming domains separated by two putative transmembrane domains. In order to clone the complete DNA sequence encoding hORK1, fragments corresponding to 5′ and 3′ sequences were isolated from fetal brain Marathon Ready cDNA (Clontech) using a rapid analysis of cDNA ends (RACE) procedure according to manufacturers instructions. The oligos used to clone 5′ and 3′ fragments were defined by the DNA sequence encoding the ORF, allowing for a 150 bp overlap between 5′ and 3′ fragments.

Oligos used in the RACE procedure:

| | |
|---|---|
| for 5′ fragment CGC AGG CAG AGC CAC AAA GAG TAC ACA G | [SEQ ID NO:41] |
| for 3′ fragment GGA GAT CAG CTA GGC ACC ATA TTT GG | [SEQ ID NO:42] |

The full length hORK1 ORF fragment was isolated and cloned into the Invitrogen TA cloning kit according to manufacturers instructions. DNA sequence analysis confirmed the presence of a single ORF sufficient to encode a protein of 426 amino acids. The complete amino acid and DNA sequences are as follows:

[SEQ ID NO:45]

MLPSASRERPGYRAGVAAPDLLDPKSAAQNSKPRLSFSTKPTVLASRVESDTTTNVMKWKTVSTIFLVVVLYLJIGATVFKALEQPTIEISQRTTJVIQKQTF
ISQHSCVNSTELDELIQQIVAAJNAGIIPLGNTSNQISHWDLGSSFFFAGTVITTIGFGNISPRTEGGKIFCIIYALLGIPLFGFLLAGVGDQLGTIFGKGI
AKVEDTFIKWNVSQTKIRIISTIIFILFGCVLFVALPAIIFKHIEGWSALDAIYFVVITLTTIGFGDYVAGGSDIEYLDFYKPVVWFWILVGLAYFAAVLSM
IGRLVRVJSKKTKEEVGEFRAHAAEWTANVTAEFKETRRRLSVEIYDRFQRATSIKRKLSAELAGNHNQELTPCRRTLSVNHLTSERDVLPPLLKTESIYLNG
LAPIICAGEEIAVJENIK

[SEQ ID NO:46]

ccatcctaatacgactcactatagggctcgagcgnccgcccgggcagtaaaatgcctgcccgtgcagctcggagcgcgcagcccgtctctgaataagaagtga
gtacaatggcgtgtttgtaaaaaaaagcttcaagtccgtctttttcaaaaaacattttgaatgctgcatgcctcATGCTTCCCAGCGCCTCGCGGGAGAGACC
CGGCTATAGAGCAGGAGTGGCGGCACCTGACTTGCTGGATCCTAAATCTGCCGCTCAGAACTCCAAACCGAGGCTCTCATTTTCCACGAAACCCACAGTGCTT
GCTTCCCGGGTGGAGAGTGACACGACCATTAATGTTATGAAATGGAAGACGGTCTCCACGATATTCCTGGTGGTTGTCCTCTATCTGATCATCGGAGCCACCG
TGTTCAAAGCATTGGAGCAGCCTCATGAGATTTCACAGAGGACCACCATTGTGATCCAGAAGCAAACATTCATATCCCAACATTCCTGTGTCAATTCGACGGA
GCTGGATGAACTCATTCAGCAAATAGTGGCAGCAATAAATGCAGGGATTATACCGTTAGGAAACACCTCCAATCAAATCAGTCACTGGGATTTGGGAAGTTCC
TTCTTCTTTGCTGGCACTGTTATTACAACCATAGGATTTGGAAACATCTCACCACGCACAGAAGGCGGCAAAATATTCTGTATCATCTATGCCTTACTGGGAA
TTCCCCTCTTTGGTTTTCTCTTGGCTGGAGTTGGAGATCAGCTAGGCACCATATTTGGAAAAGGAATTGCCAAAGTGGAAGATACGTTTATTAAGTGGAATGT
TAGTCAGACCAAGATTCGCATCATCTCAACAATCATATTTATACTATTTGGCTGTGTACTCTTTGTGGCTCTGCCTGCGATCATATTCAAACACATAGAAGGC
TGGAGTGCCCTGGACGCCATTTATTTTGTGGTTATCACTCTAACAACTATTGGATTTGGTGACTACGTTGCAGGTGGATCCGATATTGAATATCTGGACTTCT
ATAAGCCTGTCGTGTGGTTCTGGATCCYFGTAGGGCTTGCTTACTTTGCTGCTGTCCTGAGCATGATTGGGAGATTGGTCCGAGTGATATCTAAAAAGACAAA
AGAAGAGGTGGGAGAGTTCAGAGCACACGCTGCTGAGTGGACAGCCAACGTCACAGCCGAATTCAAAGAAACCAGGAGGCGACTGAGTGTGGAGATTTATGAC
AAGTTCCAGCGGGCCACCTCCATCAAGCGGAAGCTCTCGGCAGAACTGGCTGGAAACCACAATCAGGAGCTGACTCCTTGTAGGAGGACCCTGTCAGTGAACC
ACCTGACCAGCGAGAGGGATGTCTTGCCTCCCTTACTGAAGACTGAGAGTATCTATCTGAATGGTTTGGCGCCACACTGTGCTGGTGAAGAGATTGCTGTGAT
TGAGAACATCAAATAGccctctctttaaataaccttaggcatagccataggtgaggacttctctatgctctttatgactgttgctggtagcattttttaaatt
gtgcatgagctcaaagggggaacaaaatagatacacccatcatggtcatctatcatcaagagaatttggaattctgagccagcactttctttctgatgatgct
tgttgaacggcccactttctttgatgagtggaatgacaagcaatgtctgatgcctttgtgtgcccagactgttttcctctctctttccctaatgtgccataag
gcctcagaatgaattgagaattgtttctggtaacaatgtagctttgagggatcagttcttaacttttcagggtctacctaactgagcctagatatggaccatt
tatggatgacaacaattttttttttgtaaatgacaagaaattcttatgcagccttttacctaagaaatttctgtcagtgccttatcttatgaagaaacagaac
ctctctagctaatgtgtggtttctccttccctgcccccacccctaggctcacctctgcagtcttttaccccagttctcccatttgaataccataccttgntgg
aaacagngtgtaaaatgactgaagtgatgatgccgaagatgaaatagatgncaaattagntggacattga

The hORK1 ORF was amplified using oligos that added restriction endonuclease cleavage sites appropriate for insertion into the yeast expression vectors pLP100 and pYES2 (Invitrogen). The corresponding hORK1 expression plasmids, pLP155 and pLP156, were constructed using standard molecular biological methodology and used to transform S. cerevisiae CY162 cells using the lithium acetate method. The resulting yeast strains were examined for their ability to grow on standard synthetic agar media containing a low concentration of KCl. Expression of hORK1 in CY162 cells supports their growth on low (2-3 mM KCl) potassium media. Growth was observed to be more extensive when hORK1 was expressed under control of the ADHl promoter (pLP155) than with the GAL1/10 promoter (pLP156). The growth of hORK1-containing CY162 cells was inhibited by the known potassium channel blockers Ba²⁺, Ca²⁺, Cs⁺, and quinine, but not by TEA. The oligos used for the cloning of 5′ and 3′ RACE fragments were used in this analysis as well.

Oligos used to clone the hORK1 ORF into pLP100:

[SEQ ID NO:47]

5′ AAA AGA TCT AAA ATG CTT CCC AGC GCC

[SEQ ID NO:48]

3′ AAA GTC GAC CTA TTT GAT GTT CTC AAT
Oligos used to clone the hORK1 ORF into pYES2:

[SEQ ID NO:49]

5′ AAA AAG CTT AAA ATG CTT CCC AGC GCC

[SEQ ID NO:50]

3′ AAA TCT AGA CTA TTT GAT GTT CTC AAT

Northern blotting analysis of hORK1 expression in human tissues indicates that a 3.5 kb mRNA is expressed predominately in brain. The hORK1 transcript was not detected in heart, placenta, lung, liver, kidney or pancreas. Analysis of blots containing RNA from separate regions of the brain was examined and further localized high levels of hORK1 expression in the caudate nucleus, amygdala, putamen, frontal lobe, hippocampus, and spinal cord. The hORK1 transcript is present at significantly lower levels in other regions of the brain; cerebellum, cerebral cortex, medulla, occipital lobe, temporal lobe, corpus callosum, substantia nigra, subthalamic nucleus, and thalamus.

### EXAMPLE 16

2P Channels Obtained by Searching the EST Database.

The GENBANK expressed sequence tag database (dbEST) was searched for putative 2P channel coding sequences using the program TBLASTN to compare all open reading frames to the amino acid sequence of hORK1. Several sequences corresponding to TWIK were identified. In addition, one human and five murine cDNA sequences different than TWIK were identified. The five cDNAs were purchased (ATCC, Genome Systems Inc.) and subjected to automated DNA sequence analysis.

A predicted open reading frame found in partial human cDNA sequence (GENBANK accession #n39619) apparently encodes a portion of a unique putative 2P channel. DNA sequence analysis of the purchased cDNA clone (277113, SEQ ID NO:51) revealed the presence of a single long open reading frame:

AACAAAAACCTTTTTTGTTTTGAATGGCCTAGAGAGGGTAAGGGATCCCC
TGACGAACAGGAGCAGAGCCAGCTAGAACCTGGGCCTGGCCAGTTCAAGG
CCACCAGAGGGCAGCCTTCTGCGGAAGGCAGTATTGGGGTAGGCAGGGAC
CCCAGCAGACATGGCACTCAGAGCTCTCACTGTCCACTGACTCTCTCTTC
TCCAGGTTATGGCCACATGGCCCCACTATCGCCAGGCGGAAAGGCCTTCT
GCATGGTCTTANTAGCCCTTGGGCTGCCAGCCTCCTTAGCTCTCGTGGCC
ACCCTGCGCCATTGCCTGCTGCCTGTGCTCAGCCGCCCACGTGCCTGGGT
AGCGGTCCACTGGCAGCTGTCACCGGCCAGGGCTGCGCTGCTGCAGGCAG
TTGCACTGGGACTGCTGGTGGCCAGCAGCTTTGTGCTGCTGCCAGCGCTG
GTGCTGTGGGGCCTTCAGGGCGACTGCAGCCTGCTGGGGGCCGTCTACTT
CTGCTTCAGCTCGCTCAGCACCATTGGCCTGGGG

Four overlapping murine cDNA sequences (w09160, w36852, w36914, w99136) contain a predicted open reading frame sufficient to encode a portion of a unique putative 2P channel. DNA sequence analysis of the purchased cDNA clones (303895, 421453, 334194, 421453) revealed the presence of amino acid motifs corresponding to pore forming domains, transmembrane domains, and Z₄X₁X₂X₃GX₄PX₅ consensus sequences:

[EQ ID NO:52]

ATGATACGATTTAATACGACTCACTATAGGGAATTTGGCCCTCGAGGCCA
AGAATTCGGCACGAGGAGAATGTGCGCACGTTGGCTCTCATCGTGTGCAC
CTTCACCTACCTGCTGGTGGGCGCCGCGGTGTTCGACGCACTGGAGTCGG
AGCCGGAGATGATCGAGCGGCAGCGGCTGGAGCTGCGGCAGCTGGAGCTG
CGGGCGCGCTACAACCTCAGCGAGGGCGGCTACGAGGAGCTGGAGCGCGT
CGTGCTGCGCCTCAAGCCGCACAAGGCCGGCGTGCAGTGGCGCTTCGCCG
GCTCCTTCTACTTCGCCATCACCGTCATCACCACCATCGGCTATGGTCAT
GCGGCGCCCAGCACGGACGGAGGCAAGGTGTTCTGCATGTTCTACGCGCT
GCTGGGCATCCCGCTCACACTAGTCATGTTCCAGAGCCTGGGTGAACGCA
TCAACACCTCCGTGAGGTACCTGCTGCACCGTGCCAAGAGGGGGCTGGGC
ATGCGGCACGCCGAAGTGTCCATGGCCAACATGGTGCTCATCGGTTTCGT
GTCGTGCATCAGCACGCTGTGCATCGGCGCAGCTGCCTTCTCCTACTACG
AGCGCTGGACTTTCTTCCAGGCCTATTACTACTGCTTCATCACCCTCACC
ACCATCGGCTTCGGCGACTATGTGGCGCTGCAGAAGGACCAGGCGCTGCA
GACGCAGCCGCAGTATGTGGCTTCAGCTTCGTGTACATCCTCACGGGCTC
ACGGTCATCGGCGCTTCCTCAACCTCGTGGTGCTGCGATTCATGACCATG
AACGCCGAGGACGAGAAGCGTGATGCGGAGCACCGCGCCCTGCTCACGCA
CAACGGCCAGGCTGTCGGCCTGGGTGGCCTGAGCTGCCTGAGCGGTAGCC
TGGGCGACGGCGTGCGTCCCCGCGACCCAGTCACATGCGCTGCGGCCGCA
AGCTTA

[EQ ID NO:55]

gly ile trp pro ser arg pro arg lie arg his glu
glu asn val arg thr leu ala leu ile val cys thr
phe thr tyr leu leu val gly ala ala val phe asp
ala leu glu ser glu pro glu met ile glu arg gln
arg leu gln leu arg gln leu glu leu arg ala arg
tyr asn leu ser glu gly gly tyr glu glu leu glu
arg val val leu arg leu lys pro his lys ala gly
val gln trp arg phe ala gly ser phe tyr phe ala
ile thr val ile thr thr ile gly tyr gly his ala
ala pro ser thr asp gly gly lys val phe cys met
phe cys met phe tyr ala leu leu gly ile pro leu
thr leu val met phe gln ser leu gly glu arg ile
asn thr ser val arg tyr leu leu his arg ala lys
arg gly leu gly met arg his ala glu val ser met
ala asn met val leu ile gly phe val ser cys ile
ser thr leu cys ile gly ala ala ala phe ser tyr
tyr gln arg trp thr phe phe gln ala tyr tyr tyr
cys phe ile thr leu thr thr ile gly phe gly asp
tyr val ala leu gln lys asp gln ala leu gln thr
gln pro gln tyr val ala ser ala ser cys thr ser
ser arg ala his gly his arg arg phe leu asn leu
val val leu arg phe met thr met asn ala glu asp
glu lys arg asp ala gln his arg ala leu leu thr
his asn gly gln ala val gly leu gly gly leu ser
cys leu ser gly ser leu gly asp gly val arg pro
arg asp pro val thr cys ala ala ala ala ser leu
GIWPSRPRIRHEENVRTLALIVCTFTYLLVGAAGFDALESEPEMIERQRL
ELRQLELRARYNLSEGGYEELERVVLRLKPHKAGVQWRFAGSFYFAITVI
TTIGYGHAAPSTDGGKVFCMFYALLGIPLTLVMFQSLGERINTSVRYLLH
RAKRGLGMRHAEVSMANMVLIGFVSCISTLCIGAAAFSYYERWTFFQAYY
YCFITLTTIGFGDYVALQKDQALQTQPQYVASASCTSSRAHGHRRFLNLV
VLRFMTMNAEDEKRDAEHRALLTHNGQAVGLGGLSCLSGSLGDGVRPRDP
VTCAAAASL

Tissue distribution of mRNA expression determined by northern blotting analysis using a probe constituting a fragment of the open reading frame indicated high level expression in heart tissue.

A predicted open reading frame found in partial murine cDNA sequence (GENBANK accession #w18545) apparently encodes a portion of a unique putative 2P channel. DNA sequence analysis of the purchased cDNA clone (333546) revealed the presence of a single long open reading frame:

[EQ ID NO:53]

CTGAAACCATGGGCCCGATACCTGCTCCTGCTTATGGCCCACCTGCTGGC
CATGGGCCTTGGGGCTGTGGTGCTTCAGGCCCTGGAGGGCCCTCCAGCTC
GCCACCTCCAGGCCCAGGTCCAGGCTGAACTGGCTAGCTTCCAGGCAGAG
CACAGGGCCTGCTTGCCACCTGAGGCCCTGGAGGAGCTGCTAGGTGCGGT
CCTGAGAGCACAGGCCCATGGAGTTTCCAGCCTGGGCAACAGCTCANAGA
CAAGCAACTGGGATCTGCCCTCAGCTCTGCTGTTCACTGCCAGCATCCTC
ACCACCACCGGTTATGGCCACATGGCCCCACTCTCCTCAGGTGGAAAGGC
CTTCTGTGTGGTCTATGCAGCCCTTGGGCTGCCAGCCTCTCTAGCACTTG
TGGCTGCCCTGCGCCACTGCTTGCTGCCTGTGTTCAGTCGCCCAGGTGAC
TGGGTAGCCATTCGCTGGCAGCTGGCACCAGCTCAGGCTGCTCTGCTACA
GGCAGCAGGACTGGGCCTCCTGGTGGCCTGTGTCTTCATGCTGCTGCCAG
CACTGGTGCTGTGGGGTGTACAGGGTGACTGGCAGCCTGCTANAACCATC
TACTTCTGTTTCGGCTCACTCAGCACGATCGGCCTAGGAGACTTGCTGCC
TGCCCATGGACGTGGCCTGCACCCAGCCATTTACCACCTTGGGCAGTTTG
CACTTCTTGGTTACTTGCTCCTGGGGCTCCTGGCCATGTTGTTAGCAGTA
GAGACCTTCTCAGAGCTGCCTCAGGTCCGTGCCATGGTGAAATTCTTTGG
GCCCAGTGGCTCTAGAACCGATGAAGATCAAGATGGCATCCTAGGCCAAG
ATGAGCTGGCTCTGAGCACTGTGCTGCCTGACGCCCCAGTCTTGGGACCA
ACCACCCCAGCCTGAGCGGGAGGCACCAAGGAGTGCTTGAAGAACATAGC
ANGAAGGGTTATGGGAATGAATATGTCATGGGATAATGTTAATTTTAAAA
ATTAAATGGGCTGCTTAGCATGCAAAAAAAAAAAAAAAAAAAAAAAAAAA
AAAAA

The predicted translation product contains amino acid motifs corresponding to pore forming domains, transmembrane domains, and Z₄X₁X₂X₃GX₄PX₅ consensus sequences:

[SEQ ID NO:56]
leu lys pro trp ala arg tyr leu leu leu leu met ala his leu leu ala met gly leu gly ala val
val leu gln ala leu glu gly pro pro ala arg his leu gln ala gln val gln ala glu leu ala ser
phe gln ala glu his arg ala cys leu pro pro glu ala leu glu glu len leu gly ala val len arg
ala gln ala his gly val ser ser leu gly asn ser ser xxx thr ser asn trp asp leu pro ser ala
leu leu phe thr ala ser ile leu thr thr thr gly tyr gly his met ala pro leu ser ser gly gly
lys ala phe cys val val tyr ala ala leu gly leu pro ala ser leu ala leu val ala ala leu arg
his cys leu leu pro val phe ser arg pro gly asp trp val ala ile arg trp gln leu ala pro ala
gln ala ala leu leu gln ala ala gly leu gly leu leu val ala cys val phe met leu leu pro ala
leu val leu trp gly val gln gly asp trp gln pro ala xxx thr ile tyr phe cys phe gly ser leu
ser thr ile gly leu gly asp leu leu pro ala his gly arg gly leu his pro ala ile tyr his leu
gly gln phe ala leu leu gly tyr leu leu leu gly leu leu ala met leu leu ala val glu thr phe
ser glu leu pro gln val arg ala met val lys phe phe gly pro ser gly ser arg thr asp glu asp
gln asp gly ile leu gly gln asp glu leu ala leu ser thr val leu pro asp ala pro val leu gly
pro thr thr pro ala
LKQPWARYLLLLMAHLLAMGLGAVVLQALEGPPARHLQAQVQAELASFQAEHRACLPPEALEELLGAVLRAQAHGVSSLGNSSXTSNWDLP
SALLFTASILTTTGYGHMAPLSSGGKAFCVVYAALGLPASLALVAALRHCLLPVFSRPGDWVAIRWQLAPAQAALLQAAGLGLLVACVFML
LPALVLWGVQGDWQPAXTIYFCFGSLSTIGLGDLLPAHGRGLHPAIYHLGQFALLGYLLLGLLAMLLAVETFSELPQVRAMVKFFGPSGSR
TDEDQDGJLGQDELALSTVLPDAPVLGPTTPA

What is claimed is:

1. A potassium channel comprising four hydrophobic domains capable of forming transmembrane helices, wherein
(i) a first pore-forming domain is interposed between a first and a second transmembrane helix; and
(ii) a second pore-forming domain is interposed between a third and a fourth transmembrane helix.

2. The potassium channel of claim 1 wherein each pore-forming domain comprises a potassium selective peptide motif selected from the group consisting of dipeptide motifs and tripeptide motifs.

3. The potassium channel of claim 2 wherein the peptide motif comprises GXG wherein X is selected from the group of amino acids V, L, Y, F, M, or I.

4. The potassium channel of claim 3 wherein the pore-forming domain comprises ZXXZ₁Z₂Z₄GXG wherein
(i) Z through Z₂ are amino acid residues comprising T or S;
(ii) Z₃ is an amino acid residue comprising I or V; and
(iii) X is an amino acid residue comprising V, L, Y, F, M, or I.

5. The potassium channel of claim 4 where X is L or I.

6. The potassium channel of claims 1, 2, 3, 4, or 5 wherein at least one pore-forming domain is positioned proximal to an exterior portion of a cell membrane.

7. The potassium channel of claim 5 further comprising an amino acid motif ZX₁X₂X₃GX₄PX₅ downstream of said first pore-forming domain.

8. The potassium channel of claim 7 wherein ZX₁X₂X₃GX₄PX₅ is positioned about 12-25 amino acids downstream of said first pore-forming domain.

9. The potassium channel of claim 8 wherein ZX₁X₂X₃GX₄PX₅ is positioned within the second transmembrane domain.

10. The potassium channel of claim 8 or 9 wherein ZX₁X₂X₃GX₄PX₅ is positioned beginning about 16 amino acids downstream of said first pore-forming domain.

11. The potassium channel of claim 8, 9 or 10 wherein a second ZX₁X₂X₃GX₄PX₅ peptide is located within said second pore-forming region.

12. The potassium channel of claim 8, 9, or 10 wherein ZX¹ X₂X₃ comprises the amino acids YALL.

13. The potassium channels of claim 12 wherein ZX₁X₂X₃GX₄P comprises the amino acids YALLGIP.

14. The potassium channel of claim 4 further comprising a glycosylation site.

15. The potassium channel of claim 14 wherein said glycosylation site is asparagine-linked.

16. The potassium channel of claims 1, 2, 3, 4, 5, 7, or 8 characterized in that it is derived from invertebrates.

17. The potassium channel of claim 16 characterized in that it is insect-derived.

18. The potassium channel of claim 16 characterized in that it is nematode-derived.

19. The potassium channel of claims 1, 2, 3, 4, 5, 6, 7, or 8 characterized in that it is derived from vertebrates.

20. The potassium channel of claim 19 characterized in that it is mammalian derived.

21. The potassium channel of claim 20 characterized in that it is human derived.

22. An isolated nucleotide sequence capable of encoding a protein designated CORK.

23. An isolated nucleotide sequence capable of encoding a protein designated hORK.

24. An isolated nucleotide sequence comprising
(i) a nucleotide sequence depicted in SEQ ID NO 1 or 36;
(ii) a nucleotide sequence that hybridizes to said sequence depicted in SEQ ID NO:1 or 36;
(iii) a nucleotide sequence that is degenerate to the nucleotide sequence depicted in SEQ ID NO: 1 or 36; and
(iv) a functional derivative of the nucleotide sequence depicted in SEQ ID NO:1 or 36.

25. An isolated nucleotide sequence comprising
(i) a nucleotide sequence depicted in SEQ ID NO:46;
(ii) a nucleotide sequence that hybridizes to said sequence depicted in SEQ ID NO:46;
(iii) a nucleotide sequence that is degenerate to the nucleotide sequence depicted in SEQ ID NO:46; and
(iv) a functional derivative of the nucleotide sequence depicted in SEQ ID NO:46.

26. An isolated nucleotide sequence comprising (i) a nucleotide sequence depicted in SEQ ID NO:51, 52 or 53;
(ii) a nucleotide sequence that hybridizes to said sequence depicted in SEQ ID NO:51, 52 or 53;
(iii) a nucleotide sequence that is degenerate to the nucleotide sequence depicted in SEQ ID NO:51, 52 or 53; and
(iv) a functional derivative of the nucleotide sequence depicted in SEQ ID NO:52, 52, or 53.

27. An expression vector capable of expressing the potassium channel of claim 16 in a cell membrane of a yeast cell.

28. An expression vector capable of expressing the potassium channel of claim 19 in a cell membrane of a yeast cell.

29. An expression vector capable of expressing the potassium channel encoded by the nucleotide sequence of claims 24, 25, or 26 in a cell membrane of a yeast cell.

30. A transformed yeast cell comprising the expression vector of claims 27, 28, or 29.

31. A method of assaying substances to determine effects on cell growth, the method comprising the steps of:
a) preparing cultures of yeast cells in a medium adequate to support growth of potassium-dependent mutant strains expressing the potassium channel of claim 1;
b) contacting said substance to a portion of said yeast cells thereafter permitting sufficient time for continued growth, if any, of the portion of yeast cells so contacted as well as the portion not contacted with said substance;
c) identifying zones of growth around the substances, wherein the level of growth indicates whether or not activity of the heterologous potassium channel has been modulated as compared to yeast cells not contacted with said substances.

32. The method of claim 31 wherein said yeast cells comprise the nucleotide sequence of claims 24, 25, or 26.

33. A kit comprising the nucleotide sequences of claim 32.

34. A method of modulating the activity of the potassium channel of claim 19, positioned in a cellular membrane of a living organism by contacting said cellular membrane with a substance, in an amount and for a period of time sufficient to modify the ability of potassium ions to pass through said channel positioned in said cellular membrane of the living organism.

35. A method of modulating cardiac activity, by applying to a patient in need of such cardiac modulation, a substance capable of interacting with a potassium channel contained in the cardiac cells of such patient that is biologically equivalent to the potassium channel encoded by SEQ ID NO: 1 or 46, and modulating the activity of same.

36. The potassium channel of claim 7 capable of rectifying the inward and outward flow of ions.

37. The potassium channel of claim 7 capable of rectifying the outward flow of ions.

38. The potassium channel of claim 36 or 37 wherein direction and magnitude of potassium current is modulated by external potassium in concentration.

39. The potassium channel of claim 36 or 37 wherein potassium is the permeant ion.

40. A method of chromosome mapping comprising
(i) providing PCR primers from the nucleotide sequence of claims 24, 25, or 26;
(ii) performing a PCR assay of somatic cell hybrids containing chromosomes using the primers of step i); and
(iii) detecting amplified fragments as a measure of the hybrids containing the gene corresponding to the primers.

41. A transgenic animal comprising the nucleotide sequence of claims 24, 25, or 26.


SEQUENCE LISTING (56 sequences)

SEQ ID NO: 1 - DNA, 2441 bp; single-stranded, linear
  CDS 190..2043
acgcgatcgccgcgagtgtatattttttttttagctcagtcttcagtgtttcgcgattctctttaaaagaaaaaaaaaataataagtcaaaactacaaaccacacagcgaaaggcgaaagcaacggttcctgcgagtgtttattttttttttcaacaatttttgatcgtagtgcgacaatccgtcgagcatgtcgccgaatcgatggatcctgctgctcatcttctacatatcctacctgatgttcggggcggcaatctattaccatattgagcacggcgaggagaagatatcgcgcgccgaacagcgcaaggcgcaaattgcaatcaacgaatatctgctggaggagctgggcgacaagaatacgaccacacaggatgagattcttcaacggatctcggattactgtgacaaaccggttacattgccgccgacatatgatgatacgccctacacgtggaccttctaccatgccttcttcttcgccttcaccgtttgctccacggtgggatatgggaatatatcgccaaccaccttcgccggacggatgatcatgatcgcgtattcggtgattggcatccccgtcaatggtatcctctttgccggcctcggcgaatactttggacgtacgtttgaagcgatctacagacgctacaaaaagtacaagatgtccacggatatgcactatgtcccgccgcagctgggattgatcaccacggtggtgattgccctgattccgggaatagctctcttcctggtgctgccctgcgtgggtgttcacctacttcgagaactgggcctatcttccatctcgctgtactacagctatgtgaccaccacaacaattggattcggtgactatgtgcccacatttggagccaaccagcccaaggagttcggcggctggttcgtggtctatcagatctttgtgatcgtgtggttcatcttctcgctgggatatcttgtgatgatcatgacatttatcactcggggcctccagagcaagaagctggcatacctggagcagcagttgtcctccaacctgaaggccacacagaatcgcatctggtctggcgtcaccaaggatgtgggctacctccggcgaatgctcaacgagctgtacatcctcaaagtgaagcctgtgtacaccgatgtagatatcgcctacacactgccacgttccaattcgtgtccggatctgagcatgtaccgcgtggagccggctcccattcccagccggaagagggcattctccgtgtgcgccgacatggttggcgcccaaagggaggcgggcatggtacacgccaattccgatacggatctaaccaaactggatcgcgagaagacattcgagacggcggaggcgtaccaccagaccaccgatttgctggccaaggtggtcaacgcactggccacggtgaagccaccgccggcggaacaggaagatgcggctctctatggtggctatcatggcttctccgactcccagatcctggccagcgaatggtcgttctcgacggtcaacgagttcacatcaccgcgacgtccaagagcacgtgcctgctccgatttcaatctggaggcacctcgctggcagagcgagaggccactgcgttcgagccacaacgaatggacatggagcggcgacaaccagcagatccaggaggcattcaaccagcgctacaagggacagcagcgtgccaacggagcagccaactcgaccatggtccatctggagccggatgctttggaggagcagctgagaaacaatcaccgggtgccggtcgcgtcaagaagttctccatgccggatggtctgcgacgtctgtttcccttccagaagaagcacccctcgcaggatctggagcgcaagttgtccgtggtctcggtacccgagggtgtcatctcgcaggaagccagatccccgctggactactacatcaacacggtcacggcggcctccagtcaatcctatttgcgcaacggacgcggtccgccaccgcccttcgaatcgaatggcagcttggccagcggcggcggcgggctaacgaacatgggcttccagatggaggatggagcaaccccgccatcggcattgggcggtggagcctatcaacgcaaggcggctgctggcaagcgccgacgcgagagcatctacacccagaatcaagccccatccgctcgccggggcagcatgtatccgccgaccgcgcacgccttggcccagatgcagatgcgacgcggcagcttggcaaccagtggctctggatcggcggccatggcggcagtggccgcgcgtcgtggcagcctcttcccagctacagcatcggcatcatcgctgacctctgctccgcgccgaagcagcatattctcggttacctccgaaaaggatatgaatgtgctggagcagacgaccattgcggatctgattcgtgcgctcgag

SEQ ID NO: 2 - protein, 618 aa
MSPNRWILLLIFYISYLMFGAAIYYHIEHGEEKISRAEQRKAQIAINEYLLEELGDKNTTTQDEILQRISDYCDKPVTLPPTYDDTPYTWTFYHAFFFAFTVCSTVGYGNISPTTFAGRMIMIAYSVIGIPVNGILFAGLGEYFGRTFEAIYRRYKKYKMSTDMHYVPPQLGLITTVVIALIPGIALFLVLPCVGVHLLRELGLSSISLYYSYVTTTTIGFGDYVPTFGANQPKEFGGWFVVYQIFVIVWFIFSLGYLVMIMTFITRGLQSKKLAYLEQQLSSNLKATQNRIWSGVTKDVGYLRRMLNELYILKVKPVYTDVDIAYTLPRSNSCPDLSMYRVEPAPIPSRKRAFSVCADMVGAQREAGMVHANSDTDLTKLDREKTFETAEAYHQTTDLLAKVVNALATVKPPPAEQEDAALYGGYHGFSDSQILASEWSFSTVNEFTSPRRPRARACSDFNLEAPRWQSERPLRSSHNEWTWSGDNQQIQEAFNQRYKGQQRANGAANSTMVHLEPDALEEQLRNNHRVPVASRSSPCRMVCDVCFPSRRSTPRRIWSASCPWSRYPRVSSRRKPDPRWTTTSTRSRRPPVNPICATDAVRHRPSNRMAAWPAAAAG

SEQ ID NO: 3 - DNA, 1011 bp; single-stranded, linear
  CDS 1..1008
atgtccgatcagctgtttgtcgcatttgagaagtatttcttgacgagtaacgaggtcaagaagaatgcagcaacggagacatggacattttcatcgtccattttctttgccgtaaccgtcgtcactaccatcggatacggtaatccagttccagtgacaaacattggacggatatggtgtatattgttctccttgcttggaatacctctaacactggttaccatcgctgacttggcaggtaaattcctatctgaacatcttgtttggttgtatggaaactatttgaaattaaaatatctcatattgtcacgacatcgaaaagaacggagagagcacgtttgtgagcactgtcacagtcatggaatggggcatgatatgaatatcgaggagaaaagaattcctgcattcctggtattagctattctgatagtatatacagcgtttggcggtgtcctaatgtcaaaattagagccgtggtctttcttcacttcattctactggtccttcattacaatgactactgtcgggtttggcgacttgatgcccagaagggacggatacatgtatatcatattgctctatatcattttaggtaaattttcaatgaaaaaaaaacaaaaattcaaaatatttttaggtcttgcaataactacaatgtgcattgatttggtaggagtacagtatattcgaaagattcattatttcggaagaaaaattcaagacgctagatctgcattggcggttgtaggaggaaaggtagtccttgtatcagaactctacgcaaatttaatgcaaaagcgagctcgtaacatgtcccgagaagcttttatagtggagaatctctatgtttccaaacacatcataccattcataccaactgatatccgatgtattcgatatattgatcaaactgccgatgctgctaccatttccacgtcatcgtctgcaattgatatgcaaagttgtagattttgtcattcaagatattctctcaatcgtgcattcaaatag

SEQ ID NO: 4 - protein, 336 aa
MSDQLFVAFEKYFLTSNEVKKNAATETWTFSSSIFFAVTVVTTIGYGNPVPVTNIGRIWCILFSLLGIPLTLVTIADLAGKFLSEHLVWLYGNYLKLKYLILSRHRKERREHVCEHCHSHGMGHDMNIEEKRIPAFLVLAILIVYTAFGGVLMSKLEPWSFFTSFYWSFITMTTVGFGDLMPRRDGYMYIILLYIILGKFSMKKKQKFKIFLGLAITTMCIDLVGVQYIRKIHYFGRKIQDARSALAVVGGKVVLVSELYANLMQKRARNMSREAFIVENLYVSKHIIPFIPTDIRCIRYIDQTADAATISTSSSAIDMQSCRFCHSRYSLNRAFK

SEQ ID NO: 5 - DNA, 51 bp; single-stranded, linear
tccattttctttgccgtaaccgtcgtcactaccatcggatacggtaatcca

SEQ ID NO: 6 - DNA, 51 bp; single-stranded, linear
tcattctactggtccttcattacaatgactactgtcgggtttggcgacttg

SEQ ID NO: 7 - protein, 24 aa
AFLFSIETQTTIGYGFRCVTDECP

SEQ ID NO: 8 - protein, 24 aa
AFLFSLETQVTIGYGFRCVTEQCA

SEQ ID NO: 9 - protein, 24 aa
AFLFFIETEATIGYGYRYITDHCP

SEQ ID NO: 10 - protein, 24 aa
AFFFAFTVCSTVGYGNISPTTFAG

SEQ ID NO: 11 - protein, 24 aa
AFWWAVVTMTTVGYGDMTPVGFWG

SEQ ID NO: 12 - protein, 24 aa
AFWYTIVTMTTLGYGDMVPETIAG

SEQ ID NO: 13 - protein, 24 aa
AFWWAGITMTTVGYGDICPTTALG

SEQ ID NO: 14 - protein, 24 aa
GLWWALVTMTTVGYGDMAPKTYIG

SEQ ID NO: 15 - protein, 24 aa
ALYFTMTCMTSVGFGNVAAETDNE

SEQ ID NO: 16 - protein, 24 aa
CVYFLIVTMSTVGYGDVYCETVLG

SEQ ID NO: 17 - protein, 24 aa
SLYTSYVTTTTIGFGDYVPTFGAN

SEQ ID NO: 18 - protein, 24 aa
AFFFAFTVCSTVGYGNISPTTFAG

SEQ ID NO: 19 - protein, 24 aa
SIFFAVTVVTTIGYGNPVPVTNTG

SEQ ID NO: 20 - protein, 24 aa
SLYTSYVTTTTIGFGDYVPTFGAN

SEQ ID NO: 21 - protein, 24 aa
SFYWSFITMTTVGFGDLMPRRDGY

SEQ ID NO: 22 - DNA, 33 bp; single-stranded, linear
ataaagcttaaaaatgtcgccgaatcgatggat

SEQ ID NO: 23 - DNA, 30 bp; single-stranded, linear
agctctagacctccatctggaagcccatgt

SEQ ID NO: 24 - DNA, 27 bp; single-stranded, linear
aaaaagcttaaaatggcacacatcacg

SEQ ID NO: 25 - DNA, 24 bp; single-stranded, linear
aaactcgagtcatacctgtggact

SEQ ID NO: 26 - DNA, 27 bp; single-stranded, linear
aaaaagcttaaaatggtcgggcaattg

SEQ ID NO: 27 - DNA, 25 bp; single-stranded, linear
aaaagcatgctcatctggatgggca

SEQ ID NO: 28 - DNA, 27 bp; single-stranded, linear
aaaaagcttaaaatggcctcggtcgcc

SEQ ID NO: 29 - DNA, 24 bp; single-stranded, linear
ttttctagactacatcgttgtctt

SEQ ID NO: 30 - DNA, 27 bp; single-stranded, linear
aaaaagcttaaaatgaatctgatcaac

SEQ ID NO: 31 - DNA, 24 bp; single-stranded, linear
aaatctagattagtcgaaactgaa

SEQ ID NO: 32 - DNA, 24 bp; single-stranded, linear
aaaaagcttaaaatgcctggcgga

SEQ ID NO: 33 - DNA, 24 bp; single-stranded, linear
aaatctagaggctacaggaagtcc

SEQ ID NO: 34 - DNA, 27 bp; single-stranded, linear
gggggtaccaaaatgtcggggtgtgat

SEQ ID NO: 35 - DNA, 25 bp; single-stranded, linear
tttttctagatcaagagttatcatc

SEQ ID NO: 36 - DNA, 1394 bp; single-stranded, linear
atggtaataatcaaccgatcgaacacctatgccgttgagcaggaagcatttccaagagacaagtacaatattgtctactggctcgtcattcttgttggattcggagttcttctgccatggaatatgttcattactatcgcccctgagtattatgtgaattattggttcaaaccggatggcgtggagacatggtattcgaaagaattcatgggatctttgacgattggctcacaacttccaaacgcaagcattaatgttttcaacctgttcctcattattgctggtcccctgatctaccgcgtctttgctccggtttgcttcaacatcgtcaacctgacaatcattctcatcctcgtcattgttctggagcccactgaagattccatgtcctggtttttctgggtaactcttggaatggcgacttcaatcaattttagcaatgggctatatgaaaactcggtttatggagttggtggcgattttccgcacacctacattggcgctctcttgattggaaacaacatttgcggattgctgataacggttgtgaaaatcggagtgacctattttctgaatgatgagcctaaacttgttgcaatcgtctatttcggcatatcgttggtgatccttctggtgtgtgcaattgcacttttctttatcacaaagcaagatttctaccactatcaccatcaaaaaggaatggaaattcgcgaaaaggcggaaaccgacagaccgtctccatccattctttggaccacattcacaaactgttatgggcaactcttcaatgtttggttctgctttgccgttactctcacaatcttccctgttatgatgaccgttaccactcgtggagattccggcttcctaaacaaaattatgtctgaaaacgatgaaatctacactttgctcacaagtttcctcgtcttcaatttgttcgctgcgattggatccatagttgcttccaagattcactggccgacaccccgttacctcaaatttgccataatcttgcgtgctcttttcattccattcttcttcttctgcaactatcgtgtccagacgcgtgcttatcctgttttctttgagtctactgacatttttgtgattggtggaattgccatgtctttttcacatggatacctcagcgctctggcaatgggatacactccaaacgtcgtgccatctcactactcaagatttgccgctcagctttccgtttgcactcttatggttggccttctcaccggtggcctgtggcccgttgttattgagcacttcgtggacaagccaagtatcttataaatatttatagcattagagtatacttgttatatgttgtttttattaagctgtggaataaaataattattaaaaaaaaaaaaaaaaaaaaa

SEQ ID NO: 37 - protein, 479 aa
MSPNRWILLLIFYISYLMFGAAIYYHIEHGEEKISRAEQRKAQIAINEYLLEELGDKNTTTQDEILQRISDYCDKPVTLPPTYDDTPYTWTFYHAFFFAFTVCSTVGYGNISPTTFAGRMIMIAYSVIGIPVNGILFAGLGEYFGRTFEAIYRRYKKYKMSTDMHYVPPQLGLITTVVIALIPGIALFLVLPCVGVHLLRELGLSSISLYYSYVTITTIGFGDYVPTFGANQPKEFGGWFVVYQIFVIVWFIFSLGYLVMIMTFITRGLQSKKLAYLEQQLSSNLKATQNRIWSGVTKDVGYLRRMLNELYILKVKPVYTDVDIAYTLPRSNSPLSMYRVEPAPIPSRKRAFSVCADMVGAQREAGMVHANSDTDLTKLDREKTFETAEAYHQTTDLLAKVVNALATVKPPPALQEDAALYGGYHGFSDSQILASEWSFSTVNEFTSPRRPRARACSDFNLEAPRWQSERPLRSSHNEW

SEQ ID NO: 38 - protein, 335 aa
MSDQLFVAFEKYFLTSNEVKKNAATETWTFSSSIFFAVTVVTTIGYGNPVPVTNIGRIWILFSLIGIPLTLVTIALAGKFLSEHLVWLYGNYLKLKYLILSRHRKERREHVCEHCHSHGMGHDMNIEEKRIPAFLVLAILIVYTAFGGVLMSKLEPWSFFTSFYWSFITMTTVGFGDLMPRRDGYMYIILLYIILGKFSMKKKQKFKIFLGLAITTMCIDLVGVQYIRKIHYFGRKIQDARSALAVVGGKVVLVSELYANLMQKRARNMSREAFIVENLYVSKHIIPFIPTDIRCIRYIDQTADAATISTSSSAIDMQSCRFCHSRYSLNRAFKX

SEQ ID NO: 39 - DNA, 21 bp; single-stranded, linear
tnggatatctggatgactatt

SEQ ID NO: 40 - DNA, 29 bp; single-stranded, linear
agtcatccagataactccagtactagtgt

SEQ ID NO: 41 - DNA, 28 bp; single-stranded, linear
cgcaggcagagccacaaagagtacacag

SEQ ID NO: 42 - DNA, 26 bp; single-stranded, linear
ggagatcagctaggcaccatatttgg

SEQ ID NO: 43 - DNA, 26 bp; single-stranded, linear
atgctgcatgcctcatgcttcccagc

SEQ ID NO: 44 - DNA, 20 bp; single-stranded, linear
ggttatttaaagagagggct

SEQ ID NO: 45 - protein, 426 aa [sequence omitted]

SEQ ID NO: 46 - DNA, 2130 bp; single-stranded, linear
ccatcctaatacgactcactatagggctcgagcgnccgcccgggcagtaaaatgcctgcccgtgcagctcggagcgcgcagcccgtctctgaataagaagtgagtacaatggcgtgtttgtaaaaaaaagcttcaagtccgtctttttcaaaaaacattttgaatgctgcatgcctcatgcttcccagcgcctcgcgggagagacccggctatagagcaggagtggcggcacctgacttgctggatcctaaatctgccgctcagaactccaaaccgaggctctcattttccacgaaacccacagtgcttgcttcccgggtggagagtgacacgaccattaatgttatgaaatggaagacggtctccacgatattcctggtggttgtcctctatctgatcatcggagccaccgtgttcaaagcattggagcagcctcatgagatttcacagaggaccaccattgtgatccagaagcaaacattcatatcccaacattcctgtgtcaattcgacggagctggatgaactcattcagcaaatagtggcagcaataaatgcagggattataccgttaggaaacacctccaatcaaatcagtcactgggatttgggaagttccttcttctttgctggcactgttattacaaccataggatttggaaacatctcaccacgcacagaaggcggcaaaatattctgtatcatctatgccttactgggaattcccctctttggttttctcttggctggagttggagatcagctaggcaccatatttggaaaaggaattgccaaagtggaagatacgtttattaagtggaatgttagtcagaccaagattcgcatcatctcaacaatcatatttatactatttggctgtgtactctttgtggctctgcctgcgatcatattcaaacacatagaaggctggagtgccctggacgccatttattttgtggttatcactctaacaactattggatttggtgactacgttgcaggtggatccgatattgaatatctggacttctataagcctgtcgtgtggttctggatccttgtagggcttgcttactttgctgctgtcctgagcatgattgggagattggtccgagtgatatctaaaaagacaaaagaagaggtgggagagttcagagcacacgctgctgagtggacagccaacgtcacagccgaattcaaagaaaccaggaggcgactgagtgtggagatttatgacaagttccagcgggccacctccatcaagcggaagctctcggcagaactggctggaaaccacaatcaggagctgactccttgtaggaggaccctgtcagtgaaccacctgaccagcgagagggatgtcttgcctcccttactgaagactgagagtatctatctgaatggtttggcgccacactgtgctggtgaagagattgctgtgattgagaacatcaaatagccctctctttaaataaccttaggcatagccataggtgaggacttctctatgctctttatgactgttgctggtagcattttttaaattgtgcatgagctcaaagggggaacaaaatagatacacccatcatggtcatctatcatcaagagaatttggaattctgagccagcactttctttctgatgatgcttgttgaacggcccactttctttgatgagtggaatgacaagcaatgtctgatgcctttgtgtgcccagactgttttcctctctctttccctaatgtgccataaggcctcagaatgaattgagaattgtttctggtaacaatgtagctttgagggatcagttcttaacttttcagggtctacctaactgagcctagatatggaccatttatggatgacaacaattttttttttgtaaatgacaagaaattcttatgcagccttttacctaagaaatttctgtcagtgccttatcttatgaagaaacagaacctctctagctaatgtgtggtttctccttccctgcccccacccctaggctcacctctgcagtcttttaccccagttctcccatttgaataccataccttgntggaaacagngtgtaaaatgactgaagtgatgatgccgaagatgaaatagatgncaaattagntggacattga

SEQ ID NO: 47 - DNA, 27 bp; single-stranded, linear
aaaagatctaaaatgcttcccagcgcc

SEQ ID NO: 48 - DNA, 27 bp; single-stranded, linear
aaagtcgacctatttgatgttctcaat

SEQ ID NO: 49 - DNA, 27 bp; single-stranded, linear
aaaaagcttaaaatgcttcccagcgcc

SEQ ID NO: 50 - DNA, 27 bp; single-stranded, linear
aaatctagactatttgatgttctcaat

SEQ ID NO: 51 - DNA, 533 bp; single-stranded, linear
aacaaaaaccttttttgttttgaatggcctagagagggtaagggatcccctgacgaacaggagcagagccagctagaacctgggcctggccagttcaaggccaccagagggcagccttctgcggaaggcagtattggggtaggcagggaccccagcagacatggcactcagagctctcactgtccactgactctctcttctccaggttatggccacatggccccactatcgccaggcggaaaggccttctgcatggtcttatagcccttgggctgccagcctccttagctctcgtggccaccctgcgccattgcctgctgcctgtgctcagccgcccacgtgcctgggtagcggtccactggcagctgtcaccggccagggctgcgctgctgcaggcagttgcactgggactgctggtggccagcagctttgtgctgctgccagcgctggtgctgtggggccttcagggcgactgcagcctgctgggggccgtctacttctgcttcagctcgctcagcaccattggcctgggg

SEQ ID NO: 52 - DNA, 956 bp; single-stranded, linear
atgatacgatttaatacgactcactatagggaatttggccctcgaggccaagaattcggcacgaggagaatgtgcgcacgttggctctcatcgtgtgcaccttcacctacctgctggtgggcgccgcggtgttcgacgcactggagtcggagccggagatgatcgagcggcagcggctggagctgcggcagctggagctgcgggcgcgctacaacctcagcgagggcggctacgaggagctggagcgcgtcgtgctgcgcctcaagccgcacaaggccggcgtgcagtggcgcttcgccggctccttctacttcgccatcaccgtcatcaccaccatcggctatggtcatgcggcgcccagcacggacggaggcaaggtgttctgcatgttctacgcgctgctgggcatcccgctcacactagtcatgttccagagcctgggtgaacgcatcaacacctccgtgaggtacctgctgcaccgtgccaagagggggctgggcatgcggcacgccgaagtgtccatggccaacatggtgctcatcggtttcgtgtcgtgcatcagcacgctgtgcatcggcgcagctgccttctcctactacgagcgctggactttcttccaggcctattactactgcttcatcaccctcaccaccatcggcttcggcgactatgtggcgctgcagaaggaccaggcgctgcagacgcagccgcagtatgtggcttcagcttcgtgtacatcctcacgggctcacggtcatcggcgcttcctcaacctcgtggtgctgcgattcatgaccatgaacgccgaggacgagaagcgtgatgcggagcaccgcgccctgctcacgcacaacggccaggctgtcggcctgggtggcctgagctgcctgagcggtagcctgggcgacggcgtgcgtccccgcgacccagtcacatgcgctgcggccgcaagctta

SEQ ID NO: 53 - DNA, 1052 bp; single-stranded, linear
ctgaaaccatgggcccgatacctgctcctgcttatggcccacctgctggccatgggccttggggctgtggtgcttcaggccctggagggccctccagctcgccacctccaggcccaggtccaggctgaactggctagcttccaggcagagcacagggcctgcttgccacctgaggccctggaggagctgctaggtgcggtcctgagagcacaggcccatggagtttccagcctgggcaacagctcaagacaagcaactgggatctgccctcagctctgctgttcactgccagcatcctcaccaccaccggttatggccacatggccccactctcctcaggtggaaaggccttctgtgtggtctatgcagcccttgggctgccagcctctctagcacttgtggctgccctgcgccactgcttgctgcctgtgttcagtcgcccaggtgactgggtagccattcgctggcagctggcaccagctcaggctgctctgctacaggcagcaggactgggcctcctggtggcctgtgtcttcatgctgctgccagcactggtgctgtggggtgtacagggtgactggcagcctgctaaaccatctacttctgtttcggctcactcagcacgatcggcctaggagacttgctgcctgcccatggacgtggcctgcacccagccatttaccaccttgggcagtttgcacttcttggttacttgctcctggggctcctggccatgttgttagcagtagagaccttctcagagctgcctcaggtccgtgccatggtgaaattctttgggcccagtggctctagaaccgatgaagatcaagatggcatcctaggccaagatgagctggctctgagcactgtgctgcctgacgccccagtcttgggaccaaccaccccagcctgagcgggaggcaccaaggagtgcttgaagaacatagcagaagggttatgggaatgaatatgtcatgggataatgttaattttaaaaattaaatgggctgcttagcatgcaaaaaaaaaaaaaaaaaaaaaaaaaaaaaaaa

SEQ ID NO: 54 - protein, 178 aa
NKNLFCFEWPREGKGSPDQEEQSQLEPGPGQFKATRGQPSAEGSIGVGRDPSRHGTQSSHCPLTLSSPGYGHMAPLSPGGKAFCMVLXALGLPASLALVATLRHCLLPVLSRPRAWVAVHWQLSPARAALLQAVALGLLVASSFVLLPALVLWGLQGDCSLLGAVYFCFSSLSTIGLG

SEQ ID NO: 55 - protein, 312 aa
GIWPSRPRIRHEENVRTLALIVCTFTYLLVGAAVFDALESEPEMIERQRLELRQLELRARYNLSEGGYEELERVVLRLKPHKAGVQWRFAGSFYFAITVITTIGYGHAAPSTDGGKVFCMFCMFYALLGIPLTLVMFQSLGERINTSVRYLLHRAKRGLGMRHAEVSMANMVLIGFVSCISTLCIGAAAFSYYERWTFFQAYYYCFITLTTIGFGDYVALQKDQALQTQPQYVASASCTSSRAHGHRRFLNLVVLRFMTMNAEDEKRDAEHRALLTHNGQAVGLGGLSCLSGSLGDGVRPRDPVTCAAAASL

SEQ ID NO: 56 - protein, 304 aa
LKPWARYLLLLMAHLLAMGLGAVVLQALEGPPARHLQAQVQAELASFQAEHRACLPPEALEELLGAVLRAQAHGVSSLGNSSXTSNWDLPSALLFTASILTTTGYGHMAPLSSGGKAFCVVYAALGLPASLALVAALRHCLLPVFSRPGDWVAIRWQLAPAQAALLQAAGLGLLVACVFMLLPALVLWGVQGDWQPAXTIYFCFGSLSTIGLGDLLPAHGRGLHPAIYHLGQFALLGYLLLGLLAMLLAVETFSELPQVRAMVKFFGPSGSRTDEDQDGILGQDELALSTVLPDAPVLGPTTPA